(19) United States
(12) Patent Application Publication
(10) Pub. No.: US 2017/0360929 A1
(43) Pub. Date: Dec. 21, 2017

(54) STABLE AQUEOUS ANTIBODY FORMULATION FOR ANTI TNF ALPHA ANTIBODIES

(71) Applicant: PFIZER INC., NEW YORK, NY (US)

(72) Inventors: Sandipan SINHA, Chesterfield, MO (US); Mariko AOKI, Chesterfield, MO (US)

(73) Assignee: PFIZER INC., NEW YORK, NY (US)

(21) Appl. No.: 15/539,529

(22) PCT Filed: Dec. 9, 2015
(86) PCT No.: PCT/IB2015/059481

§ 371 Date: Jun. 23, 2017

Related U.S. Application Data
(60) Provisional application No. 62/096,452, filed on Dec. 23, 2014.

(51) Int. Cl. A61K 39/395; A61K 47/22; A61K 47/26; A61K 47/18; C07K 16/24; A61K 47/12; A61K 39/00; A61K 9/00

(52) U.S. Cl. CPC A61K 39/39591; C07K 16/241; A61K 47/22; A61K 47/12; A61K 47/26; A61K 47/183; A61K 9/0019; A61K 2039/505

(57) ABSTRACT

The present invention relates to the field of pharmaceutical formulations of antibodies. Specifically, the present invention relates to a stable liquid antibody formulation comprising methionine and its pharmaceutical preparation and use. This invention is exemplified by a liquid formulation of an anti-Tumor Necrosis Factor alpha (TNFα) antibody.

### CROSS-REFERENCE TO RELATED APPLICATIONS

This application claims the benefit of U.S. Provisional Application No. 62/096,452 filed Dec. 23, 2014, which is hereby incorporated by reference in its entirety.

### FIELD OF THE INVENTION

The present invention relates to the field of pharmaceutical formulations of antibodies. Specifically, the present invention relates to a stable liquid antibody formulation comprising methionine and its pharmaceutical preparation and use.

### BACKGROUND

Antibody preparations intended for therapeutic or prophylactic use require stabilizers to prevent loss of activity or structural integrity of the protein due to the effects of denaturation, oxidation or aggregation over a period of time during storage and transportation prior to use. These problems are exacerbated at the high concentrations of antibody often desired for therapeutic administration.

A major aim in the development of antibody formulations is to maintain antibody, solubility, stability and potency of its antigen binding. It is particularly desirable to avoid aggregates and particulates in solution which would require sterile filtration before use for intravenous or subcutaneous injection and limit route of administration. Formulation of antibody preparations requires careful selection of these factors among others to avoid denaturation of the protein and loss of antigen-binding activity. Accordingly, there is a need for a stable aqueous antibody formulation which stably supports high concentrations of bioactive antibody in solution and is suitable for parenteral administration, including intravenous, intramuscular, intraperitoneal, intradermal, or subcutaneous injection.

Furthermore there is a need to provide such a stable aqueous formulation for an anti-TNFα antibody. It has been shown that the TNFα antibody is useful in the treatment of, for example, rheumatoid arthritis, juvenile idiopathic arthritis, psoriatic arthritis, ankylosing spondylitis, Crohn's disease, ulcerative colitis, and plaque psoriasis (see, e.g., U.S. Pat. Nos. 6,090,382, 8,889,135, and 8,889,136). There is a need for a stable aqueous antibody preparation of an anti-TNFα antibody to meet the medical need of patients suffering from conditions mediated by TNFα. U.S. Pat. No. 8,216,583 describes a stable aqueous antibody formulation comprising a human anti-TNFα antibody.

All publications, patents, and patent applications cited herein are hereby incorporated by reference herein in their entirety for all purposes to the same extent as if each individual publication, patent, and patent application were specifically and individually indicated to be so incorporated by reference. In the event that one or more of the incorporated literature and similar materials differs from or contradicts this application, including but not limited to defined terms, term usage, described techniques, or the like, this application controls.

### SUMMARY OF THE INVENTION

Stable aqueous pharmaceutical formulations with an extended shelf life comprising an anti-tumor necrosis factor alpha (TNFα) antibody are provided. It is demonstrated that the aqueous pharmaceutical formulation of the present invention with high antibody concentration is stable (e.g., having low levels of % HMMS (High Molecular Mass Species), % (LMMS (Low Molecular Mass species), % fragment, and oxidation) and suitable for parenteral administration.

In one aspect, provided is an aqueous formulation comprising: a. 35 mg/ml to about 200 mg/ml of an anti-Tumor Necrosis Factor alpha (TNFα) antibody, or antigen-binding fragment thereof; a buffer; a polyol; methionine; a surfactant; a chelating agent; and wherein the formulation has a pH at about 5.0 to about 6.0.

In some embodiments, the buffer is a histidine buffer. In some embodiments, the concentration of the buffer is about 1 mM to about 100 mM.

In some embodiments, the polyol is sucrose. In some embodiments, the concentration of the polyol is about 1 mg/mL to about 300 mg/ml.

In some embodiments, the surfactant is a polysorbate, such as polysorbate 20 or 80. In some embodiments, the concentration of the surfactant is about 0.01 mg/ml to about 10 mg/ml.

In some embodiments, the chelating agent is disodium EDTA dihydrate (disodium edetate dihydrate). In some embodiments, the concentration of the chelating agent is about 0.01 mg/ml to about 1.0 mg/ml.

In some embodiments, the antibody, or the antigen-binding fragment thereof, comprises a heavy chain variable region (VH) complementarity determining region one (CDR1) having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 3 or 12, or a variant of SEQ ID NO: 3 having a single alanine substitution at position 2, 3, 4, 5, 6, 8, 9, 10, or 11, or by one to five conservative amino acid substitutions at positions 2, 3, 4, 5, 6, 8, 9, 10, 11, and/or 12, a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 6 or 13, or a variant of SEQ ID NO: 6 having a single alanine substitution at position 1, 4, 5, 7, or 8, or by one to five conservative amino acid substitutions at positions 1, 3, 4, 6, 7, 8, and/or 9. In some embodiments, the antibody, or the antigen-binding fragment thereof, comprises a heavy chain variable region (VH) and a light chain variable region (VL), wherein the VH region comprises the amino acid sequence of SEQ ID NO: 7, and the VL region comprises the amino acid sequence of SEQ ID NO: 8. In some embodiments, the antibody is a human antibody, such as adalimumab (HUMIRA® or D2E7, see e.g., U.S. Pat. Nos. 6,090,382 and 8,216,583). In some embodiments, the antibody, or the antigen-binding fragment thereof, comprises the heavy chain variable region CDR1, CDR2, and CDR3 of adalimumab (e.g., SEQ ID NOs: 1, 2, and 3, respectively), and the light chain variable region CDR1, CDR2, and CDR3 of adalimumab (e.g., SEQ ID NOs: 4, 5, and 6, respectively). In some embodiments, the concentration of the antibody, or the antigen-binding fragment thereof, is 35 mg/mL, 40 mg/mL, 45 mg/ml, 50 mg/mL, 55 mg/mL, or 60 mg/mL.

In another aspect, provided is an aqueous formulation comprising: about 50 mg/ml of an anti-Tumor Necrosis Factor alpha (TNFα) protein, or an antigen-binding fragment thereof; about 20 mM histidine buffer; about 85 mg/mL sucrose; about 0.2 mg/mL methionine; about 0.2 mg/ml polysorbate 80; about 0.05 mg/ml disodium EDTA; wherein the antibody, or the antigen-binding fragment thereof, comprises a heavy chain variable region comprising the amino acid sequence of SEQ ID NO: 7, and a light chain variable region comprising the amino acid sequence of SEQ ID NO: 8; and wherein the formulation has pH at 5.5. In some embodiments, the antibody is adalimumab (HUMIRA® or D2E7).

In another aspect, provided is a method for treating or inhibiting a TNFα related disorder in a subject in need thereof, comprising administering to the subject a therapeutically effective amount of the formulation as described herein. In some embodiments, the formulation is administered to the subject subcutaneously or intravenously.

In another aspect, provided is a use of the formulation as described herein for the manufacture of a medicament for treatment of a TNFα related disorder in a subject.

In some embodiments, the TNFα related disorder is selected from the group consisting of rheumatoid arthritis, juvenile idiopathic arthritis, axial spondyloarthritis, psoriatic arthritis, ankylosing spondylitis, axial spondyloarthritis without radiographic evidence of ankylosing spondylitis, Crohn's disease (e.g., adult), pediatric Crohn's disease, ulcerative colitis, psoriasis arthropathica, intestinal Behcet's disease, plaque psoriasis, and hidradenitis suppurativa.

In some embodiments, the formulation as described herein has a shelf life of at least about 24 months.

### BRIEF DESCRIPTION OF THE DRAWINGS

FIGS. 1A-1B show high molecular mass species (% HMMS) by SE-HPLC (Size Exclusion-High Performance Liquid Chromatography) for adalimumab commercial product (“Adalimumab-EU”), adalimumab in commercial formulation, and adalimumab in PF formulation at 25° C. (FIG. 1A) and at 40° C. (FIG. 1B).

FIGS. 2A-2B show level of fragments by r-CGE (reducing-capillary gel electrophoresis) for adalimumab commercial product (“Adalimumab-EU”), adalimumab in commercial formulation, and adalimumab in PF formulation at 25° C. (FIG. 2A) and at 40° C. (FIG. 2B).

FIGS. 3A-3B show level of methionine-253 (Met-253) oxidation for adalimumab commercial product (“Adalimumab-EU”), adalimumab in commercial formulation, and adalimumab in PF formulation at 25° C. (FIG. 3A) and at 40° C. (FIG. 3B).

FIGS. 4A-4B show level of acidic species by iCE (Imaged Capillary Electrophoresis) for adalimumab commercial product (“Adalimumab-EU”), adalimumab in commercial formulation, and adalimumab in PF formulation at 25° C. (FIG. 4A) and at 40° C. (FIG. 4B).

FIGS. 5A-5B show level of basic species by iCE for adalimumab commercial product (“Adalimumab-EU”), adalimumab in commercial formulation, and adalimumab in PF formulation at 25° C. (FIG. 5A) and at 40° C. (FIG. 5B).

FIGS. 6A-6B show comparison of SE-HPLC chromatograms of adalimumab commercial product (“Adalimumab-EU”), adalimumab in commercial formulation, and adalimumab in PF formulation after storage at 25° C. for 6 months (FIG. 6A) and at 40° C. for 3 months (FIG. 6B).

FIGS. 6C-6D show comparison of iCE electropherograms of adalimumab commercial product (“Adalimumab-EU”), adalimumab in commercial formulation, and adalimumab in PF formulation after storage at 25° C. for 6 months (FIG. 6C) and at 40° C. for 3 months (FIG. 6D).

FIGS. 6E-6F show comparison of rCGE electropherograms of adalimumab commercial product (“Adalimumab-EU”), adalimumab in commercial formulation, and adalimumab in PF formulation after storage at 25° C. for 6 months (FIG. 6E) and at 40° C. for 3 months (FIG. 6F).

FIGS. 6G-6H show comparison of chromatograms for Met-253 oxidation of adalimumab commercial product (“Adalimumab-EU”), adalimumab in commercial formulation, and adalimumab in PF formulation after storage at 25° C. for 6 months (FIG. 6G) and at 40° C. for 3 months (FIG. 6H).

### DETAILED DESCRIPTION

Disclosed herein are stable aqueous pharmaceutical formulations with an extended shelf-life comprising an anti-tumor necrosis factor alpha (TNFα) antibody. It is demonstrated that the aqueous pharmaceutical formulation of the present invention stably supports high concentration of antibody (e.g., having low levels of % HMMS (High Molecular Mass Species), % LMMS (Low Molecular Mass species), % fragment, and oxidation at an antibody concentration of at least 35 mg/mL) and is suitable for parenteral administration, including subcutaneous, intravenous, intramuscular, intraperitoneal, or intradermal injection. Accordingly, in one aspect, provided is an aqueous formulation comprising: about 35 mg/ml to about 200 mg/ml of an anti-Tumor Necrosis Factor alpha (TNFα) antibody, or antigen-binding fragment thereof; a buffer; a polyol; methionine; a surfactant; a chelating agent; and wherein the formulation has a pH at about 5.0 to about 6.0. For example, in some embodiments, provided is an aqueous formulation comprising: about 35 mg/ml to about 200 mg/ml of an anti-Tumor Necrosis Factor alpha (TNFα) antibody, or antigen-binding fragment thereof; (e.g., adalimumab); about 1 mM to about 100 mM of a buffer (e.g., histidine buffer); about 1 mg/mL to about 300 mg/mL of a polyol (e.g., sucrose); about 0.01 mg/mL to about 10 mg/mL of methionine; about 0.01 mg/ml to about 10 mg/ml of a surfactant (e.g., polysorbate 80); about 0.01 mg/ml to about 1.0 mg/ml of a chelating agent (e.g., disodium EDTA dihydrate (or disodium edetate dihydrate)); wherein the formulation has a pH at about 5.0 to about 6.0. In some embodiments, the antibody concentration is about 50 mg/mL.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

### Definitions

The following terms, unless otherwise indicated, shall be understood to have the following meanings: the term “isolated molecule” (where the molecule is, for example, a polypeptide, a polynucleotide, or an antibody) is a molecule that by virtue of its origin or source of derivation (1) is not associated with naturally associated components that accompany it in its native state, (2) is substantially free of other molecules from the same species (3) is expressed by a cell from a different species, or (4) does not occur in nature. Thus, a molecule that is chemically synthesized, or expressed in a cellular system different from the cell from which it naturally originates, will be “isolated” from its naturally associated components. A molecule also may be rendered substantially free of naturally associated components by isolation, using purification techniques well known in the art. Molecule purity or homogeneity may be assayed by a number of means well known in the art. For example, the purity of a polypeptide sample may be assayed using polyacrylamide gel electrophoresis and staining of the gel to visualize the polypeptide using techniques well known in the art. For certain purposes, higher resolution may be provided by using HPLC or other means well known in the art for purification.

As used herein, the term “formulation” as it relates to an antibody is meant to describe the antibody in combination with a pharmaceutically acceptable excipient comprising at least one buffer, at least one stabilizer, methionine, at least one surfactant, at least one chelating agent, and wherein the pH is as defined.

The terms “pharmaceutical composition” or “pharmaceutical formulation” refer to preparations which are in such form as to permit the biological activity of the active ingredients to be effective.

“Pharmaceutically acceptable excipients” (vehicles, additives) are those, which can safely be administered to a subject to provide an effective dose of the active ingredient employed. The term “excipient” or “carrier” as used herein refers to an inert substance, which is commonly used as a diluent, vehicle, preservative, binder or stabilizing agent for drugs. As used herein, the term “diluent” refers to a pharmaceutically acceptable (safe and non-toxic for administration to a human) solvent and is useful for the preparation of the aqueous formulations herein. Exemplary diluents include, but are not limited to, sterile water and bacteriostatic water for injection (BWFI).

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also, unless otherwise specified, any antigen binding portion thereof that competes with the intact antibody for specific binding, fusion proteins comprising an antigen binding portion, and any other modified configuration of the immunoglobulin molecule that comprises an antigen recognition site. Antigen binding portions include, for example, Fab, Fab′, F(ab′)2, Fd, Fv, domain antibodies (dAbs, e.g., shark and camelid antibodies), fragments including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR (i.e., in the framework region), appropriate amino acid substitution, preferably, conservative amino acid substitution, can be identified by comparing the subject variable region to the variable regions of other antibodies which contain CDR1 and CDR2 sequences in the same canonincal class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, and the conformational definition.

The Kabat definition is a standard for numbering the residues in an antibody and is typically used to identify CDR regions. See, e.g., Johnson & Wu, 2000, Nucleic Acids Res., 28: 214-8. The Chothia definition is similar to the Kabat definition, but the Chothia definition takes into account positions of certain structural loop regions. See, e.g., Chothia et al., 1986, J. Mol. Biol., 196: 901-17; Chothia et al., 1989, Nature, 342: 877-83. The AbM definition uses an integrated suite of computer programs produced by Oxford Molecular Group that model antibody structure. See, e.g., Martin et al., 1989, Proc Natl Acad Sci (USA), 86:9268-9272; “ABM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., 1999, “Ab lnitio Protein Structure Prediction Using a Combined Hierarchical Approach,” in PROTEINS, Structure, Function and Genetics Suppl., 3:194-198. The contact definition is based on an analysis of the available complex crystal structures. See, e.g., MacCallum et al., 1996, J. Mol. Biol., 5:732-45. In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

As known in the art, a “constant region” of an antibody refers to the constant region of the antibody light chain or the constant region of the antibody heavy chain, either alone or in combination.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. The monoclonal antibodies may also be isolated from phage libraries generated using the techniques described in McCafferty et al., 1990, Nature 348:552-554, for example. As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

A “human antibody” is one which possesses an amino acid sequence which corresponds to that of an antibody produced by a human and/or has been made using any of the techniques for making human antibodies as disclosed herein. This definition of a human antibody specifically excludes a humanized antibody comprising non-human antigen binding residues.

As used herein, the term “human antibody” is intended to include antibodies having variable and constant regions derived from human germline immunoglobulin sequences. This definition of a human antibody includes antibodies comprising at least one human heavy chain polypeptide or at least one human light chain polypeptide. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, “humanized” antibody refers to forms of non-human (e.g. murine) antibodies that are chimeric immunoglobulins, immunoglobulin chains, or fragments thereof (such as Fv, Fab, Fab′, F(ab′)2 or other antigen-binding subsequences of antibodies) that contain minimal sequence derived from non-human immunoglobulin. Preferably, humanized antibodies are human immunoglobulins (recipient antibody) in which residues from a complementarity determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Preferred are antibodies having Fc regions modified as described in WO 99/58572. Other forms of humanized antibodies have one or more CDRs (CDR L1, CDR L2, CDR L3, CDR H1, CDR H2, or CDR H3) which are altered with respect to the original antibody, which are also termed one or more CDRs “derived from” one or more CDRs from the original antibody.

There are four general steps to humanize a monoclonal antibody. These are: (1) determining the nucleotide and predicted amino acid sequence of the starting antibody light and heavy variable domains (2) designing the humanized antibody, i.e., deciding which antibody framework region to use during the humanizing process (3) the actual humanizing methodologies/techniques and (4) the transfection and expression of the humanized antibody. See, for example, U. S. Pat. Nos. 4,816,567; 5,807,715; 5,866,692; 6,331,415; 5,530,101; 5,693,761; 5,693,762; 5,585,089; and 6,180,370.

A number of “humanized” antibody molecules comprising an antigen-binding site derived from a non-human immunoglobulin have been described, including chimeric antibodies having rodent or modified rodent V regions and their associated complementarity determining regions (CDRs) fused to human constant domains. See, for example, Winter et al. Nature 349: 293-299 (1991), Lobuglio et al. Proc. Nat. Acad. Sci. USA 86: 4220-4224 (1989), Shaw et al. J Immunol. 138: 4534-4538 (1987), and Brown et al. Cancer Res. 47: 3577-3583 (1987). Other references describe rodent CDRs grafted into a human supporting framework region (FR) prior to fusion with an appropriate human antibody constant domain. See, for example, Riechmann et al. Nature 332: 323-327 (1988), Verhoeyen et al. Science 239: 1534-1536 (1988), and Jones et al. Nature 321: 522-525 (1986). Another reference describes rodent CDRs supported by recombinantly veneered rodent framework regions. See, for example, European Patent Publication No. 0519596. These“humanized”molecules are designed to minimize unwanted immunological response toward rodent anti-human antibody molecules which limits the duration and effectiveness of therapeutic applications of those moieties in human recipients. For example, the antibody constant region can be engineered such that it is immunologically inert (e. g., does not trigger complement lysis). See, e. g. PCT Publication No. WO99/58572; UK Patent Application No. 9809951.8. Other methods of humanizing antibodies that may also be utilized are disclosed by Daugherty et al., Nucl. Acids Res. 19: 2471-2476 (1991) and in U.S. Pat. Nos. 6,180,377; 6,054,297; 5,997,867; 5,866,692; 6,210,671; and 6,350,861; and in PCT Publication No. WO 01/27160.

As used herein, the term “recombinant antibody” is intended to include all antibodies that are prepared, expressed, created or isolated by recombinant means, for example antibodies expressed using a recombinant expression vector transfected into a host cell, antibodies isolated from a recombinant, combinatorial human antibody library, antibodies isolated from an animal (e.g., a mouse) that is transgenic for human immunoglobulin genes or antibodies prepared, such recombinant human antibodies can be subjected to in vitro mutagenesis.

The term “epitope” refers to that portion of a molecule capable of being recognized by and bound by an antibody at one or more of the antibody's antigen-binding regions. Epitopes often consist of a surface grouping of molecules such as amino acids or sugar side chains and have specific three-dimensional structural characteristics as well as specific charge characteristics. In some embodiments, the epitope can be a protein epitope. Protein epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds. The term “antigenic epitope” as used herein, is defined as a portion of an antigen to which an antibody can specifically bind as determined by any method well known in the art, for example, by conventional immunoassays. Once a desired epitope on an antigen is determined, it is possible to generate antibodies to that epitope, e.g., using the techniques described in the present specification. Alternatively, during the discovery process, the generation and characterization of antibodies may elucidate information about desirable epitopes. From this information, it is then possible to competitively screen antibodies for binding to the same epitope. An approach to achieve this is to conduct competition and cross-competition studies to find antibodies that compete or cross-compete with one another for binding to TNFα, e.g., the antibodies compete for binding to the antigen.

As used herein, the terms “isolated antibody” or “purified antibody” refers to an antibody that by virtue of its origin or source of derivation has one to four of the following: (1) is not associated with naturally associated components that accompany it in its native state, (2) is free of other proteins from the same species, (3) is expressed by a cell from a different species, or (4) does not occur in nature.

The term “antagonist antibody” refers to an antibody that binds to a target and prevents or reduces the biological effect of that target. In some embodiments, the term can denote an antibody that prevents the target, e.g., TNFα, to which it is bound from performing a biological function.

An antibody that “preferentially binds” or “specifically binds” (used interchangeably herein) to an epitope is a term well understood in the art, and methods to determine such specific or preferential binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. An antibody “specifically binds” or “preferentially binds” to a target if it binds with greater affinity, avidity, more readily, and/or with greater duration than it binds to other substances. For example, an antibody that specifically or preferentially binds to a TNFα epitope is an antibody that binds this epitope sequence with greater affinity, avidity, more readily, and/or with greater duration than it binds to other sequences. It is also understood by reading this definition that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

As used herein, “immunospecific” binding of antibodies refers to the antigen specific binding interaction that occurs between the antigen-combining site of an antibody and the specific antigen recognized by that antibody (i.e., the antibody reacts with the protein in an ELISA or other immunoassay, and does not react detectably with unrelated proteins).

The term “compete”, as used herein with regard to an antibody, means that a first antibody, or an antigen-binding portion thereof, binds to an epitope in a manner sufficiently similar to the binding of a second antibody, or an antigen-binding portion thereof, such that the result of binding of the first antibody with its cognate epitope is detectably decreased in the presence of the second antibody compared to the binding of the first antibody in the absence of the second antibody. The alternative, where the binding of the second antibody to its epitope is also detectably decreased in the presence of the first antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing and/or cross-competing antibodies are encompassed and can be useful for the methods disclosed herein.

As used herein, the term “human TNFα” refers to a human cytokine that exists as a 17 kD in secreted form and a 26 kD in membrane associated form (see, e.g., SEQ ID NO: 9). The biologically active form of the human TNFα is a trimer of noncovalently bound 17 kD molecules. See, e.g., Pennica D., et al., Nature 312:724-729 (1984), David J. M., et al., Biochemistry 26: 1322-1326 (1987), Jones, E. Y., et al. Nature 338:225-228 (1989). Human TNFα also encompasses recombinant human TNFα, which can be prepared by standard recombinant expression methods as described herein or purchased commercially (see, e.g., R&D Systems, Catalog No. 210-TA, Minneapolis, Minn.).

A “therapeutically effective amount” refers to an amount effective, at dosages and for periods of time necessary, to achieve the desired therapeutic result, which in the context of anti-TNFα antibodies includes treatment or prophylactic prevention of the targeted pathologic condition for example high blood glucose. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. Likewise, a therapeutically effective amount of the antibody or antibody portion may vary according to factors such as the disease state, age, sex, and weight of the individual, the ability of the antibody or antibody portion to elicit a desired response in the individual, and the desired route of administration of the antibody formulation. A therapeutically effective amount is also one in which any toxic or detrimental effects of the antibody or antibody portion are outweighed by the therapeutically beneficial effects.

As used herein, the term “treatment” refers to both therapeutic treatment and prophylactic or preventative measures, wherein the object is to prevent or slow down (lessen) the targeted pathologic condition (e.g., a TNFα -related disorder, including any aspect of autoimmune disease (e.g., rheumatoid arthritis, diabetes, and multiple sclerosis), infectious disease, transplantation rejection, pulmonary disorders (e.g., adult respiratory distress syndrome, shock lung, chronic pulmonary inflammatory disease, pulmonary sarcoidosis, pulmonary fibrosis, and silicosis, intestinal disorders (e.g., Crohn's disease and ulcerative colitis), and cardiac disorders (e.g., ischemia of the heart)). Those in need of treatment include those already with the condition as well as those prone to have the condition or those in whom the condition is to be prevented. As used herein, “treatment” is an approach for obtaining beneficial or desired clinical results including, but not limited to, one or more of the following: including lessening severity, alleviation of one or more symptoms associated with a TNFα-related disorder.

An “effective amount” of drug, formulation, compound, or pharmaceutical composition is an amount sufficient to effect beneficial or desired results including clinical results such as alleviation or reduction of the targeted pathologic condition. An effective amount can be administered in one or more administrations. For purposes of this invention, an effective amount of drug, compound, or pharmaceutical composition is an amount sufficient to treat, ameliorate, or reduce the intensity of the targeted pathologic condition. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, the term “subject” for purposes of treatment includes any subject, and preferably is a subject who is in need of the treatment of the targeted pathologic condition (e.g., a TNFα-related disorder). For purposes of prevention, the subject is any subject, and preferably is a subject that is at risk for, or is predisposed to, developing the targeted pathologic condition. The term “subject” is intended to include living organisms, e.g., prokaryotes and eukaryotes. Examples of subjects include mammals, e.g., humans, dogs, cows, horses, pigs, sheep, goats, cats, mice, rabbits, rats, and transgenic non-human animals. In specific embodiments of the invention, the subject is a human.

As used herein, the term “polynucleotide” or “nucleic acid”, used interchangeably herein, means a polymeric form of nucleotides either ribonucleotides or deoxynucleotides or a modified form of either type of nucleotide and may be single and double stranded forms. A “polynucleotide” or a “nucleic acid” sequence encompasses its complement unless otherwise specified. As used herein, the term “isolated polynucleotide” or “isolated nucleic acid” means a polynucleotide of genomic, cDNA, or synthetic origin or some combination thereof, which by virtue of its origin or source of derivation, the isolated polynucleotide has one to three of the following: (1) is not associated with all or a portion of a polynucleotide with which the “isolated polynucleotide” is found in nature, (2) is operably linked to a polynucleotide to which it is not linked in nature, or (3) does not occur in nature as part of a larger sequence.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline, normal (0.9%) saline, or 5% dextrose. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 20th Ed. Mack Publishing, 2000).

The term “K_{off}”, as used herein, is intended to refer to the off rate constant for dissociation of an antibody from the antibody/antigen complex.

The term “K_{d}”, as used herein, is intended to refer to the dissociation constant of an antibody-antigen interaction. One way of determining the Kd or binding affinity of antibodies to human TNFα is by measuring binding affinity of monofunctional Fab fragments of the antibody. To obtain monofunctional Fab fragments, an antibody (for example, IgG) can be cleaved with papain or expressed recombinantly. The affinity of an anti-TNFα Fab fragment of an antibody can be determined by surface plasmon resonance (BlAcorC1GM000™ surface plasmon resonance (SPR) system, BlAcore, INC, Piscaway N.J.). CM5 chips can be activated with N-ethyl-N′-(3-dimethylaminopropyl)-carbodiinide hydrochloride (EDC) and N-hydroxysuccinimide (NHS) according to the supplier's instructions.

“Reducing incidence” means any of reducing severity (which can include reducing need for and/or amount of (e.g., exposure to) other drugs and/or therapies generally used for this condition. As is understood by those skilled in the art, individuals may vary in terms of their response to treatment, and, as such, for example, a “method of reducing incidence” reflects administering the human TNFα antagonist antibody based on a reasonable expectation that such administration may likely cause such a reduction in incidence in that particular individual.

“Ameliorating” means a lessening or improvement of one or more symptoms as compared to not administering a TNFα antagonist antibody. “Ameliorating” also includes shortening or reduction in duration of a symptom.

Reference to “about” a value or parameter herein includes (and describes) embodiments that are directed to that value or parameter per se. For example, description referring to “about X” includes description of “X.” Numeric ranges are inclusive of the numbers defining the range.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

When introducing elements of the present invention or the preferred embodiment(s) thereof, the articles “a”, “an”, “the” and “said” are intended to mean that there are one or more of the elements. The terms “comprising”, “comprise”, “comprises”, “including” and “having” are intended to be inclusive and mean that there may be additional elements other than the listed elements. It is understood that wherever embodiments are described herein with the language “comprising,” otherwise analogous embodiments described in terms of “consisting of” and/or “consisting essentially of” are also provided.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the present invention. The materials, methods, and examples are illustrative only and not intended to be limiting.

### Anti-TNFα Antibody Formulation

In one aspect, provided is a stable aqueous formulation comprising: about 35 mg/ml to about 200 mg/ml of an anti-Tumor Necrosis Factor alpha (TNFα) antibody, or antigen-binding fragment thereof; a buffer; a polyol; methionine; a surfactant; a chelating agent; and wherein the formulation has a pH at about 5.0 to about 6.0. The formulation described herein have an extended shelf life, preferably of at least or more than about 24 months (e.g., at about 5° C.).

In some embodiments, the formulation comprises at least one anti-TNFα antibody. For example, the anti-TNFα antibody is a human antibody (e.g., adalimumab (HUMIRA® or D2E7)), a humanized antibody, or a chimeric antibody (e.g., infliximab or REMICADE®). In some embodiments, more than one antibody may be present. At least one, at least two, at least three, at least four, at least five, or more, different antibodies can be present. Generally, the two or more different antibodies have complementarity activities that do not adversely affect each other. The, or each, antibody can also be used in conjunction with other agents that serve to enhance and/or complement the effectiveness of the antibodies.

In some embodiments, the anti-TNFα antibody, or the antigen-binding fragment thereof, in the formulation of the present invention is an antibody that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷ M or less. In some embodiments, the anti-TNFα antibody, or the antigen-binding fragment thereof, in the formulation of the present invention is an antibody that dissociates from human TNFα with a K_{off} rate constant of 5×10⁻⁴ s⁻¹ or less, or K_{off} rate constant of 1×10⁻⁴ s⁻¹ or less. In some embodiments, the anti-TNFα antibody in the formulation of the present invention is an antibody neutralizes human TNFα cytotoxicity in a standard in vitro L929 assay with an IC₅₀ of 1×10⁻⁷ M or less, an IC₅₀ of 1×10⁻⁸ M or less, an IC₅₀ of 1×10⁻⁹ M or less, or an IC₅₀ of 1×10⁻¹⁰ M or less. In some embodiment, the anti-TNFα antibody, or the antigen-binding fragment thereof, in the formulation of the present invention also neutralizes TNFα-induced cellular activation, as assessed using a standard in vitro assay for TNFα-induced ELAM-1 expression on human umbilical vein endothelial cells (HUVEC). See, e.g., U.S. Pat. Nos. 6,090,382, 6,258,562, and 8,216,583, each incorporated by reference herein.

In some embodiments, the anti-TNFα antibody, or the antigen-binding fragment thereof, in the formulation of the present invention, comprises a heavy chain variable region (VH) complementarity determining region one (CDR1) having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 3 or 12, or a variant of SEQ ID NO: 3 having a single alanine substitution at position 2, 3, 4, 5, 6, 8, 9, 10, or 11, or by one to five conservative amino acid substitutions at positions 2, 3, 4, 5, 6, 8, 9, 10, 11, and/or 12, a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 6 or 13, or a variant of SEQ ID NO: 6 having a single alanine substitution at position 1, 4, 5, 7, or 8, or by one to five conservative amino acid substitutions at positions 1, 3, 4, 6, 7, 8, and/or 9.

In some embodiments, the anti-TNFα antibody, or the antigen-binding fragment thereof, in the formulation of the present invention, comprises a heavy chain variable region (VH) and a light chain variable region (VL), wherein the VH region comprises the amino acid sequence of SEQ ID NO: 7, and the VL region comprises the amino acid sequence of SEQ ID NO: 8. In some embodiments, the anti-TNFα antibody, or the antigen-binding fragment thereof, in the formulation of the present invention, has an IgG1 heavy chain constant region or an IgG4 heavy chain constant region, or is a Fab fragment or a single chain Fv fragment.

In some embodiments, the anti-TNFα antibody in the formulation of the present invention is adalimumab (HUMIRA®) or D2E7. In some embodiments, the anti-TNFα antibody in the formulation of the present invention comprises the heavy chain variable region CDR1, CDR2, and CDR3 of adalimumab (e.g., SEQ ID NOs: 1, 2, and 3, respectively), and the light chain variable region CDR1, CDR2, and CDR3 of adalimumab (e.g., SEQ ID NOs: 4, 5, and 6, respectively).

The antibody may be present in the formulation at a concentration ranging from about 0.1 mg/ml to about 200 mg/ml, from about 35 mg/ml to 200 mg/ml, from about 35 mg/ml to about 100 mg/ml, or from about 37 mg/ml to about 65 mg/ml. For example, in some embodiments, the concentration of antibody is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 70 mg/ml, about 80 mg/ml, about 90 mg/ml, about 100 mg/ml, about 101 mg/ml, about 102 mg/ml, about 102.5 mg/ml, about 103 mg/ml, about 103.5 mg/ml, about 104 mg/ml, about 104.5 mg/ml, about 105 mg/ml, about 105.5 mg/ml, about 106 mg/ml, about 106.5 mg/ml, about 107 mg/ml, about 107.5 mg/ml, about 108 mg/ml, about 108.5 mg/ml, about 109 mg/ml, about 109.5 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, about 150 mg/ml, about 151 mg/ml, about 152 mg/ml, about 153 mg/ml, about 154 mg/ml, about 155 mg/ml, about 156 mg/ml, about 157 mg/ml, about 158 mg/ml, about 159 mg/ml, about 160 mg/ml, about 170 mg/ml, about 180 mg/ml, about 190 mg/ml, or about 200 mg/ml.

According to the present invention, the buffer (e.g., histidine buffer) provides the formulation with a pH close to physiological pH for reduced risk of pain or anaphylactoid side effects on injection and also provides enhanced antibody stability and resistance to aggregation, oxidation, and fragmentation.

The buffer can be, for example without limitation, acetate, succinate, gluconate, citrate, histidine, acetic acid, phosphate, phosphoric acid, ascorbate, tartartic acid, maleic acid, glycine, lactate, lactic acid, ascorbic acid, imidazole, bicarbonate and carbonic acid, succinic acid, sodium benzoate, benzoic acid, gluconate, edetate, acetate, malate, imidazole, tris, phosphate, and mixtures thereof. Preferably the buffer is histidine, wherein the histidine can comprise either L-histidine or D-histidine, a solvated form of histidine, a hydrated form (e.g., monohydrate including L-histidine hydrochloride monohydrate) of histidine, a salt of histidine (e.g., histidine hydrochloride) or an anhydrous form of histidine or a mixture thereof.

The concentration of the buffer can range from about 0.1 millimolar (mM) to about 100 mM. Preferably, the concentration of the buffer is from about 0.5 mM to about 50 mM, further preferably about 1 mM to about 30 mM, more preferably about 1 mM to about 25 mM. Preferably, the concentration of the buffer is about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. In some embodiment, the buffer is a histidine buffer in the concentration of about 20 mM.

The concentration of the buffer can also range from about 0.01 mg/ml to about 10 mg/ml, from about 0.1 mg/ml to about 5 mg/ml, or from about 0.5 mg/ml to about 4 mg/ml. For example, the concentration of the buffer is about 0.01 mg/ml, 0.02 mg/ml, 0.03 mg/ml, about 0.04 mg/ml, about 0.05 mg/ml, about 0.06 mg/ml, about 0.07 mg/ml, 0.08 mg/ml, 0.09 mg/ml, about 0.10 mg/ml, 0.11 mg/ml, 0.12 mg/ml, 0.13 mg/ml, about 0.14 mg/ml, about 0.15 mg/ml, about 0.16 mg/ml, about 0.17 mg/ml, 0.18 mg/ml, 0.19 mg/ml about 0.20 mg/ml, about 0.25 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, or 10.0 mg/ml. In some embodiments, the buffer is a histidine buffer comprising about 0.5-2.0 mg/mL L-histidine and about 1-10 mg/mL L-histidine hydrochloride monohydrate, about 0.5-1.0 mg/mL L-histidine and about 1-5 mg/mL L-histidine hydrochloride monohydrate. In some embodiment, the buffer is a histidine buffer comprising about 0.786 mg/mL L-histidine and about 3.132 mg/mL L-histidine hydrochloride monohydrate.

According to the present invention, the polyol is an isotonicity modifying agent which protects the antibody or protein in the formulation against freeze-thaw induced aggregation as well as aggregation on storage. The polyol can have a molecular weight that, for example without limitation, is less than about 600 kD (e.g., in the range from about 120 to about 400 kD), and comprises multiple hydroxyl groups including sugars (e.g., reducing and nonreducing sugars or mixtures thereof, saccharide, or a carbohydrate), sugar alcohols, sugar acids, or a salt or mixtures thereof. Examples of non-reducing sugar, include, but are not limited to, sucrose, trehalose, and mixtures thereof. In some embodiments, the polyol is mannitol, trehalose, sorbitol, erythritol, isomalt, lactitol, maltitol, xylitol, glycerol, lactitol, propylene glycol, polyethylene glycol, inositol, or mixtures thereof. In other embodiments, the polyol can be, for example without limitation, a monosaccharide, disaccharide or polysaccharide, or mixtures of any of the foregoing. The saccharide or carbohydrate can be, for example without limitation, fructose, glucose, mannose, sucrose, sorbose, xylose, lactose, maltose, sucrose, dextran, pullulan, dextrin, cyclodextrins, soluble starch, hydroxyethyl starch, water-soluble glucans, or mixtures thereof.

The concentration of the polyol in the formulation ranges from about 1 mg/ml to about 300 mg/ml, from about 1 mg/ml to about 200 mg/ml, or from about 1 mg/ml to about 120 mg/ml. Preferably the concentration of the polyol in the formulation is about 50 mg/ml to about 120 mg/ml, from about 60 mg/ml to about 110 mg/ml, or from about 63 mg/ml to about 107 mg/ml (e.g., 63.75 mg/ml to about 106.25 mg/ml). For example, the concentration of the polyol in the formulation is about 0.5 mg/ml, about 1 mg/ml, about 2 mg/ml, about 2.5 mg/ml, about 3 mg/ml, about 3.5 mg/ml, about 4 mg/ml, about 4.5 mg/ml, about 5 mg/ml, about 5.5 mg/ml, about 6 mg/ml, about 6.5 mg/ml, about 7 mg/ml, about 7.5 mg/ml, about 8 mg/ml, about 8.5 mg/ml, about 9 mg/ml, about 9.5 mg/ml, about 10 mg/ml, about 11 mg/ml, about 12 mg/ml, about 13 mg/ml, about 14 mg/ml, about 15 mg/ml, about 16 mg/ml, about 17 mg/ml, about 18 mg/ml, about 19 mg/ml, about 20 mg/ml, about 21 mg/ml, about 22 mg/ml, about 23 mg/ml, about 24 mg/ml, about 25 mg/ml, about 26 mg/ml, about 27 mg/ml, about 28 mg/ml, about 29 mg/ml, about 30 mg/ml, about 31 mg/ml, about 32 mg/ml, about 33 mg/ml, about 34 mg/ml, about 35 mg/ml, about 36 mg/ml, about 37 mg/ml, about 38 mg/ml, about 39 mg/ml, about 40 mg/ml, about 41 mg/ml, about 42 mg/ml, about 43 mg/ml, about 44 mg/ml, about 45 mg/ml, about 46 mg/ml, about 47 mg/ml, about 48 mg/ml, about 49 mg/ml, about 50 mg/ml, about 51 mg/ml, about 52 mg/ml, about 53 mg/ml, about 54 mg/ml, about 55 mg/ml, about 56 mg/ml, about 57 mg/ml, about 58 mg/ml, about 59 mg/ml, about 60 mg/ml, about 65 mg/ml, about 70 mg/ml, about 75 mg/ml, about 80 mg/ml, about 81 mg/ml, about 82 mg/ml, about 83 mg/ml, about 84 mg/ml, about 85 mg/ml, about 86 mg/ml, about 87 mg/ml, about 88 mg/ml, about 89 mg/ml, about 90 mg/ml, about 91 mg/ml, about 92 mg/ml, about 93 mg/ml, about 94 mg/ml, about 95 mg/ml, about 96 mg/ml, about 97 mg/ml, about 98 mg/ml, about 99 mg/ml, about 100 mg/ml, about 101 mg/ml, about 102 mg/ml, about 103 mg/ml, about 104 mg/ml, about 105 mg/ml, about 106 mg/ml, about 107 mg/ml, about 108 mg/ml, about 109 mg/ml, about 110 mg/ml, about 111 mg/ml, about 112 mg/ml, about 113 mg/ml, about 114 mg/ml, about 115 mg/ml, about 116 mg/ml, about 117 mg/ml, about 118 mg/ml, about 119 mg/ml, about 120 mg/ml, about 121 mg/ml, about 122 mg/ml, about 123 mg/ml, about 124 mg/ml, about 125 mg/ml, about 126 mg/ml, about 127 mg/ml, about 128 mg/ml, about 129 mg/ml, about 130 mg/ml, about 131 mg/ml, about 132 mg/ml, about 133 mg/ml, about 134 mg/ml, about 135 mg/ml, about 136 mg/ml, about 137 mg/ml, about 138 mg/ml, about 139 mg/ml, about 140 mg/ml, about 141 mg/ml, about 142 mg/ml, about 143 mg/ml, about 144 mg/ml, about 145 mg/ml, about 146 mg/ml, about 147 mg/ml, about 148 mg/ml, about 149 mg/ml, or about 150 mg/ml.

In some embodiments, the polyol is sucrose at a concentration of from about 1 mg/ml to about 300 mg/ml, from about 1 mg/ml to about 200 mg/ml, or from about 1 mg/ml to about 120 mg/ml. Preferably the concentration of the sucrose in the formulation is about 50 mg/ml to about 120 mg/ml, from about 60 mg/ml to about 110 mg/ml, or from about 63 mg/ml to about 107 mg/ml (e.g., about 63.75 mg/ml to about 106.25 mg/ml). In some embodiments, the concentration of sucrose in the formulation is about 85 mg/ml.

In some embodiments, where the polyol comprises a salt, the concentration of the salt in the formulation ranges from about 1 mg/ml to about 20 mg/ml. Salts that are pharmaceutically acceptable and suitable for this invention include sodium chloride, sodium succinate, sodium sulfate, potassium chloride, magnesium chloride, magnesium sulfate, and calcium chloride. In some embodiments the salt in the formulation is selected from a range of concentrations of any of about 1 mg/ml, 2 mg/ml, 3 mg/ml, 4 mg/ml, 5 mg/ml, 6 mg/ml, 7 mg/ml, 8, mg/ml, 9 mg/ml, 10 mg/ml, 11 mg/ml, 12 mg/ml, 13 mg/ml, 14 mg/ml, 15 mg/ml, 16 mg/ml, 17 mg/ml, 18 mg/ml, 19 mg/ml and 20 mg/ml.

The formulation of the present invention also comprises methionine, which acts as a stabilizer for the antibody or the protein in the formulation. In some embodiments, the methionine is L-methionine. The concentration of the methionine can range from about 0.01 mg/ml to about 10 mg/ml, from about 0.05 mg/ml to about 5 mg/ml, from about 0.1 mg/ml to about 1 mg/ml, from about 0.1 mg/ml to about 0.5 mg/ml. In some embodiments, the concentration of the methionine is about 0.01 mg/ml, 0.02 mg/ml, 0.03 mg/ml, 0.04 mg/ml, 0.05 mg/ml, 0.06 mg/ml, 0.07 mg/ml, 0.08 mg/ml, 0.09 mg/ml, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 2 mg/ml, 3 mg/ml, 4 mg/ml, 5 mg/ml, 6 mg/ml, 7 mg/ml, 8 mg/ml, 9 mg/ml, or 10 mg/ml.

In some embodiments, the formulation can comprise an antioxidant agent, including but not limited to, sodium thiosulfate, catalase, and platinum. The concentration of the antioxidant generally ranges from about 0.01 mg/ml to about 50 mg/ml, from about 0.01 mg/ml to about 10.0 mg/ml, from about 0.01 mg/ml to about 5.0 mg/ml, from about 0.01 mg/ml to about 1.0 mg/ml, or from about 0.01 mg/ml to about 0.02 mg/ml. Preferably the concentration of the antioxidant can be about 0.01 mg/ml, 0.02 mg/ml, 0.03 mg/ml, about 0.04 mg/ml, about 0.05 mg/ml, about 0.06 mg/ml, about 0.07 mg/ml, 0.08 mg/ml, 0.09 mg/ml, about 0.10 mg/ml, 0.11 mg/ml, 0.12 mg/ml, 0.13 mg/ml, about 0.14 mg/ml, about 0.15 mg/ml, about 0.16 mg/ml, about 0.17 mg/ml, 0.18 mg/ml, 0.19 mg/ml about 0.20 mg/ml, about 0.25 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml. Most preferably, the concentration of the antioxidant is about 0.01 mg/ml.

Surfactants, as used in the present invention, can alter the surface tension of a liquid antibody formulation. In certain embodiments, the surfactant reduces the surface tension of a liquid antibody formulation. In still other embodiments, the surfactant can contribute to an improvement in stability of any of the antibody in the formulation. The surfactant can also reduce aggregation of the formulated antibody (e.g., during shipping and storage) and/or minimize the formation of particulates in the formulation and/or reduces adsorption (e.g., adroption to a container). For example, the surfactant can also improve stability of the antibody during and after a freeze/thaw cycle. The surfactant can be, for example without limitation, a polysorbate, poloxamer, triton, sodium dodecyl sulfate, sodium laurel sulfate, sodium octyl glycoside, lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine, myristamidopropyl-dimethylamine, palmidopropyl-dimethylamine, isostearamidopropyl-dimethylamine, sodium methyl cocoyl-taurate, disodium methyl oleyl-taurate, dihydroxypropyl PEG 5 linoleammonium chloride, polyethylene glycol, polypropylene glycol, and mixtures thereof. The surfactant can be, for example without limitation, polysorbate 20, polysorbate 21, polysorbate 40, polysorbate 60, polysorbate 61, polysorbate 65, polysorbate 80, polysorbate 81, polysorbate 85, PEG3350 and mixtures thereof.

The concentration of the surfactant generally ranges from about 0.01 mg/ml to about 10 mg/ml, from about 0.01 mg/ml to about 5.0 mg/ml, from about 0.01 mg/ml to about 2.0 mg/ml, from about 0.01 mg/ml to about 1.5 mg/ml, from about 0.01 mg/ml to about 1.0 mg/ml, from about 0.01 mg/ml to about 0.5 mg/ml, from about 0.01 mg/ml to about 0.4 mg/ml, from about 0.01 mg/ml to about 0.3 mg/ml, from about 0.01 mg/ml to about 0.2 mg/ml, from about 0.01 mg/ml to about 0.15 mg/ml, from about 0.01 mg/ml to about 0.1 mg/ml, from about 0.01 mg/ml to about 0.05 mg/ml, from about 0.1 mg/ml to about 1 mg/ml, from about 0.1 mg/ml to about 0.5 mg/ml, or from about 0.1 mg/ml to about 0.3 mg/ml. Further preferably the concentration of the surfactant is about 0.05 mg/ml, about 0.06 mg/ml, about 0.07 mg/ml, about 0.08 mg/ml, about 0.09 mg/ml, about 0.1 mg/ml, about 0.15 mg/ml, about 0.2 mg/ml, about 0.3 mg/ml, about 0.4 mg/ml, about 0.5 mg/ml, about 0.6 mg/ml, about 0.7 mg/ml, about 0.8 mg/ml, about 0.9 mg/ml, or about 1 mg/ml.

In some embodiments, the polysorbate is polysorbate 80 at a concentration ranging from about 0.1 mg/ml to about 0.3 mg/ml, for example, at 0.2 mg/ml.

Chelating agents, as used in the present invention, lower the formation of reduced oxygen species, reduce acidic species (e.g., deamidation) formation, reduce antibody aggregation, and/or reduce antibody fragmentation, and/or reduce antibody oxidation in the formulation of the present invention. For example, the chelating agent can be a multidentate ligand that forms at least one bond (e.g., covalent, ionic, or otherwise) to a metal ion and acts as a stabilizer to complex with species, which might otherwise promote instability.

In some embodiments, the chelating agent can be selected from the group consisting of aminopolycarboxylic acids, hydroxyaminocarboxylic acids, N-substituted glycines, 2-(2-amino-2-oxocthyl) aminoethane sulfonic acid (BES), deferoxamine (DEF), citric acid, niacinamide, and desoxycholates and mixtures thereof. In some embodiments, the chelating agent is selected from the group consisting of ethylenediaminetetraacetic acid (EDTA), diethylenetriamine pentaacetic acid 5 (DTPA), nitrilotriacetic acid (NTA), N-2-acetamido-2-iminodiacetic acid (ADA), bis(aminoethyl)glycolether, N,N,N′,N′-tetraacetic acid (EGTA), trans-diaminocyclohexane tetraacetic acid (DCTA), glutamic acid, and aspartic acid, N-hydroxyethyliminodiacetic acid (HIMDA), N,N-bis-hydroxyethylglycine (bicine) and N-(trishydroxymethylmethyl) 10 glycine (tricine), glycylglycine, sodium desoxycholate, ethylenediamine, propylenediamine, diethylenetriamine, triethylenetetraamine (trien),disodium edetate dihydrate (or disodium EDTA dihydrate or EDTA disodium salt), calcium EDTA oxalic acid, malate, citric acid, citric acid monohydrate, and trisodium citrate-dihydrate, 8-hydroxyquinolate, amino acids, histidine, cysteine, methionine, peptides, polypeptides, and proteins and mixtures thereof. In some embodiments, the chelating agent is selected from the group consisting of salts of EDTA including dipotassium edetate, disodium edetate, edetate calcium disodium, sodium edetate, trisodium edetate, and potassium edetate; and a suitable salt of deferoxamine (DEF) is deferoxamine mesylate (DFM), or mixtures thereof. Chelating agents used in the invention can be present, where possible, as the free acid or free base form or salt form of the compound, also as an anhydrous, solvated or hydrated form of the compound or corresponding salt.

Most preferably the chelating agent is disodium EDTA dihydrate (or disodium edetate dihydrate).

The concentration of the chelating agent generally ranges from about 0.01 mg/ml to about 50 mg/ml, from about 0.1 mg/ml to about 10.0 mg/ml, from about 5 mg/ml to about 15.0 mg/ml, from about 0.01 mg/ml to about 1.0 mg/ml, from about 0.02 mg/ml to about 0.5 mg/ml, from about 0.025 mg/ml to about 0.075 mg/ml. Further preferably, the concentration of the chelating agent generally ranges from about 0.01 mM to about 2.0 mM, from about 0.01 mM to about 1.5 mM, from about 0.01 mM to about 0.5 mM, from about 0.01 mM to about 0.4 mM, from about 0.01 mM to about 0.3 mM, from about 0.01 mM to about 0.2 mM, from about 0.01 mM to about 0.15 mM, from about 0.01 mM to about 0.1 mM, from about 0.01 mM to about 0.09 mM, from about 0.01 mM to about 0.08 mM, from about 0.01 mM to about 0.07 mM, from about 0.01 mM to about 0.06 mM, from about 0.01 mM to about 0.05 mM, from about 0.01 mM to about 0.04 mM, from about 0.01 mM to about 0.03 mM, from about 0.01 mM to about 0.02 mM, from about 0.02, or from about 0.05 mM to about 0.01 mM. Preferably the concentration of the chelating agent can be about 0.01 mg/ml, about 0.02 mg/ml, about 0.025 mg/ml, about 0.03 mg/ml, about 0.04 mg/ml, about 0.05 mg/ml, about 0.06 mg/ml, about 0.07 mg/ml, about 0.075 mg/ml, about 0.08 mg/ml, about 0.09 mg/ml, about 0.10 mg/ml, or about 0.20 mg/ml. Further preferably the concentration of chelating agent is about 0.025 mg/ml, about 0.03 mg/ml, about 0.035 mg/ml, about 0.04 mg/ml, about 0.045 mg/ml, about 0.05 mg/ml, about 0.055 mg/ml, about 0.06 mg/ml, about 0.065 mg/ml, about 0.07 mg/ml, or about 0.075 mg/ml. Most preferably, the concentration of the chelating agent is about 0.05 mg/ml.

According to some embodiments of the present invention, the pH can be in the range of about pH 5.0 to 8.0, preferably between about pH 5.0 to 6.5 or about 5.0 to 6.0, and most preferably between pH 5.2 to 5.8. For example, the anti-TNFα antibody in the formulation of the present invention at the pH range of 5.2 to 5.8 had less formation of low molecular mass species compared to at pH 5.0 or pH 6.5. Accordingly, in some embodiments, the pH for the formulation of the present invention can be in the range selected from between any one of about pH 5.2, 5.3, 5.4, 5.5, or 5.6 and any one of about pH 6.5, 6.4, 6.3, 6.2, 6.1, 6.0, 5.9, 5.8 or 5.7. In some embodiments the pH can be selected from pH values of any of about pH 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4 or 7.5. Preferably, the pH is pH 5.5+/−0.5, and most preferably, the pH is pH 5.5+/−0.3.

In some embodiments the formulation can comprise a preservative. Preferably the preservative agent is selected from phenol, m-cresol, benzyl alcohol, benzalkonium chloride, benzalthonium chloride, phenoxyethanol and methyl paraben.

The concentration of the preservative generally ranges from about 0.001 mg/ml to about 50 mg/ml, from about 0.005 mg/ml to about 15.0 mg/ml, from about 0.008 mg/ml to about 12.0 mg/ml or from about 0.01 mg/ml to about 10.0 mg/ml. Preferably the concentration of preservative can be about 0.1 mg/ml, 0.2 mg/ml, 0.3 mg/ml, about 0.4 mg/ml, about 0.5 mg/ml, about 0.6 mg/ml, about 0.7 mg/ml, 0.8 mg/ml, 0.9 mg/ml about 1.0 mg/ml, 2.0 mg/ml, 3.0 mg/ml, about 4.0 mg/ml, about 5.0 mg/ml, about 6.0 mg/ml, about 7.0 mg/ml, 8.0 mg/ml, 9.0 mg/ml about 9.1 mg/ml, about 9.2 mg/ml, 9.3 mg/ml, 9.4 mg/ml, 9.5 mg/ml, 9.6 mg/ml, 9.7 mg/ml, 9.8 mg/ml, 9.9 mg/ml, 10.0 mg/ml. Most preferably, the concentration of preservative is about 0.1 mg/ml or 9.0 mg/mL.

In some embodiments, the formulation does not contain a preservative.

In some embodiments, the antibody can be selected from the group consisting of monoclonal antibodies, polyclonal antibodies, antibody fragments (e.g., Fab, Fab′, F(ab′)2, Fv, Fc, ScFv etc.), chimeric antibodies, bispecific antibodies, heteroconjugate antibodies, single chain (ScFv), mutants thereof, fusion proteins comprising an antibody portion (e.g., a domain antibody), humanized antibodies, human antibodies, and any other modified configuration of the immunoglobulin molecule that comprises an antigen recognition site of the required specificity, including glycosylation variants of antibodies, amino acid sequence variants of antibodies, and covalently modified antibodies. The antibody may be murine, rat, human, or any other origin (including chimeric or humanized antibodies). In some embodiments, the antibody can be human but is more preferably humanized. Preferably the antibody is isolated, further preferably it is substantially pure. Where the antibody is an antibody fragment this preferably retains the functional characteristics of the original antibody i.e. the ligand binding and/or antagonist or agonist activity.

In some embodiments, the antibody heavy chain constant region may be from any type of constant region, such as IgG, IgM, IgD, IgA, and IgE; and any isotypes, such as IgG1, IgG2, IgG3, and IgG4. Preferably the antibody is an IgG1 antibody.

In some embodiments, the antibody can comprise the human heavy chain IgG2a constant region. In some embodiments the antibody comprises the human light chain kappa constant region. In some embodiments, the antibody comprises a modified constant region, such as a constant region that is immunologically inert, e.g., does not trigger complement mediated lysis, or does not stimulate antibody-dependent cell mediated cytotoxicity (ADCC). In other embodiments, the constant region is modified as described in Eur. J. Immunol. (1999) 29:2613-2624; PCT publication No. WO099/58572; and/or UK Patent Application No. 9809951.8. In still other embodiments, the antibody comprises a human heavy chain IgG2a constant region comprising the following mutations: A330P331 to S330S331 (amino acid numbering with reference to the wildtype IgG2a sequence), Eur. J. Immunol. (1999) 29:2613-2624.

According to a further aspect of the present invention there is provided an aqueous formulation comprising or consisting of: about 35 mg/ml to about 200 mg/ml of an anti-Tumor Necrosis Factor alpha (TNFα) antibody, or an antigen-binding fragment thereof; about 1 mM to about 100 mM of a buffer; about 1 mg/mL to about 300 mg/mL of a polyol; about 0.01 mg/mL to about 10 mg/mL of methionine; about 0.01 mg/ml to about 10 mg/ml of a surfactant; about 0.01 mg/ml to about 1.0 mg/ml of a chelating agent; and wherein the formulation has a pH at about 5.0 to about 6.0. In some embodiments, the antibody, or antigen-binding fragment thereof, comprises a heavy chain variable region (VH) CDR1 having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 3 or 12, or a variant of SEQ ID NO: 3 having a single alanine substitution at positions 2, 3, 4, 5, 6, 8, 9, 10, or 11, or by one to five conservative amino acid substitutions at positions 2, 3, 4, 5, 6, 8, 9, 10, 11, and/or 12, and a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 6 or 13, or a variant of SEQ ID NO: 6 having a single alanine substitution at positions 1, 4, 5, 7, or 8, or by one to five conservative amino acid substitutions at positions 1, 3, 4, 6, 7, 8, and/or 9. In some embodiments, the anti-TNFα antibody comprises a VH region comprising the amino acid sequence of SEQ ID NO: 7, and a VL region comprising the amino acid sequence of SEQ ID NO: 8. In some embodiments, the anti-TNFα antibody is adalimumab (HUMIRA® or D2E7). In some embodiments, the anti-TNFα antibody comprises the heavy chain variable region CDR1, CDR2, and CDR3 of adalimumab (e.g., SEQ ID NOs: 1, 2, and 3, respectively), and the light chain variable region CDR1, CDR2, and CDR3 of adalimumab (e.g., SEQ ID NOs: 4, 5, and 6, respectively).

In some embodiments, the buffer is histidine buffer, the polyol is sucrose, the surfactant is a polysorbate (e.g., polysorbate 80), and/or the chelating agent is disodium EDTA dehydrate (or disodium edetate dehydrate).

According to a further aspect of the present invention, there is provided an aqueous formulation comprising or consisting of: about 35 mg/ml, 40 mg/ml, 45 mg/ml, 50 mg/ml, 55 mg/ml, or 60 mg/ml of an anti-Tumor Necrosis Factor alpha (TNFα) antibody (e.g., human anti-TNFα antibody), or antigen-binding fragment thereof; about 1 mM to about 100 mM of a buffer; about 1 mg/mL to about 300 mg/mL of a polyol; about 0.01 mg/mL to about 10 mg/mL of methionine; about 0.01 mg/ml to about 10 mg/ml of a surfactant; about 0.01 mg/ml to about 1.0 mg/ml of a chelating agent; and wherein the formulation has a pH at about 5.0 to about 6.0. In some embodiments, the antibody, or antigen-binding fragment thereof, comprises a heavy chain variable region (VH) CDR1 having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 12, and a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 13. In some embodiments, the anti-TNFα antibody comprises a VH region comprising the amino acid sequence of SEQ ID NO: 7, and a VL region comprising the amino acid sequence of SEQ ID NO: 8. In some embodiments, the anti-TNFα antibody is adalimumab (HUMIRA® or D2E7).

According to a further aspect of the present invention there is provided an aqueous formulation comprising or consisting of: about 35 mg/ml to about 65 mg/ml of an anti-Tumor Necrosis Factor alpha (TNFα) antibody, or antigen-binding fragment thereof; about 20 mM of a buffer; about 1 mg/mL to about 300 mg/mL of a polyol; about 0.1 mg/mL to about 0.3 mg/mL of methionine; about 0.1 mg/ml to about 0.3 mg/ml of a surfactant; about 0.025 mg/ml to about 0.075 mg/ml of a chelating agent; and wherein the formulation has a pH at about 5.0 to about 6.0. In some embodiments, the antibody, or antigen-binding fragment thereof, comprises a heavy chain variable region (VH) CDR1 having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 3 or 12, or a variant of SEQ ID NO: 3 having a single alanine substitution at position 2, 3, 4, 5, 6, 8, 9, 10, or 11, or by one to five conservative amino acid substitutions at positions 2, 3, 4, 5, 6, 8, 9, 10, 11, and/or 12, and a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 6 or 13, or a variant of SEQ ID NO:6 having a single alanine substitution at position 1, 4, 5, 7, or 8, or by one to five conservative amino acid substitutions at positions 1, 3, 4, 6, 7, 8, and/or 9. In some embodiments, the anti-TNFα antibody comprises a VH region comprising the amino acid sequence of SEQ ID NO: 7, and a VL region comprising the amino acid sequence of SEQ ID NO: 8. In some embodiments, the anti-TNFα antibody is adalimumab (HUMIRA® or D2E7). In some embodiments, the buffer is a histidine buffer, the polyol is sucrose, the chelating agent is disodium EDTA dehydrate (or disodium edetate dehydrate), and/or the surfactant is polysorbate 80.

In some embodiments, provided is an aqueous formulation comprising or consisting of: about 50 mg/ml of an antagonist antibody that specifically binds to a human anti-Tumor Necrosis Factor alpha (TNFα) antibody, or antigen-binding fragment thereof; about 20 mM of histidine buffer; about 85 mg/mL of sucrose; about 0.2 mg/mL of methionine; about 0.2 mg/ml of polysorbate 80; about 0.025 mg/ml to about 0.05 mg/ml of disodium EDTA dehydrate (or disodium edetate dehydrate); and wherein the formulation has a pH at about 5.5. In some embodiments, the antibody, or antigen-binding fragment thereof, comprises a heavy chain variable region (VH) CDR1 having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 12, and a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 13. In some embodiments, the anti-TNFα antibody comprises a VH region comprising the amino acid sequence of SEQ ID NO: 7, and a VL region comprising the amino acid sequence of SEQ ID NO: 8. In some embodiments, the anti-TNFα antibody is adalimumab (HUMIRA® or D2E7).

In some embodiments, the formulation as described herein has a shelf life of at least or more than about 6 months, 12 months, 18 months, 24 months, 30 months, 36 months, 42 months, or 48 months (e.g., at 5° C., 25° C., or 40° C.). For example, in some embodiments, the formulation of the present invention has a shelf life of at least about 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, 36 months, 37 months, 38 months, 39 months, 40 months, 41 months, 42 months, 43 months, 44 months, 45 months, 46 months, 47 months, 48 months, 49 months, 50 months, 51 months, 52 months, 53 months, 54 months, 55 months, 56 months, 57 months, 58 months, 59 months, or 60 months (e.g., at 5° C., 25° C., or 40° C.).

In some embodiments, the formulation as described herein has less than about 1% HMMS at 40° C. for up to 1 month, 2 months, 3 months, 4 months, 5 months, or 6 months (e.g., as measured by size exclusion HPLC). In some embodiments, the formulation as described herein has less than about 6.5% LMMS at 40° C. for up to 1 month, 2 months, 3 months, 4 months, 5 months, or 6 months (e.g., as measured by size exclusion HPLC).

In some embodiments, the formulation as described herein has less than about 4% HMMS for up to 7 days under high intensity light conditions (e.g., as described in Example 3).

Unless stated otherwise, the concentrations listed herein are those concentrations at ambient conditions, i.e., at 25° C. and atmospheric pressure.

In some embodiments there is provided a formulation which is lyophilized and/or has been subjected to lyophylization. In some embodiments there is provided a composition which is not lyophilized and has not been subjected to lyophylization.

### Methods of Using the Anti-TNFα Antibody Formulation

The formulations of the present invention are useful in various applications including, but are not limited to, therapeutic treatment methods.

In one aspect, the invention provides a method for treating or inhibiting a TNFα -related disorder in a subject. Accordingly, in some embodiments, provided is a method of treating or inhibiting a TNFα related disorder in a subject in need thereof comprising administering to the subject a therapeutically effective amount of the formulation as described herein. In some embodiments, provided is a use of the formulation of the present invention for the manufacture of a medicament for treatment of a TNFα-related disorder in a subject.

As used herein, the TNFα-related disorder refers to a disorder in which inhibition of TNFα activity is expected to alleviate the symptoms and/or progression of such disorder. Examples of a TNFα-related disorder include, but are not limited to, autoimmune diseases/disorders, infectious diseases, intestinal disorders, pulmonary disorders, cardiac disorders, transplantation rejection, sepsis, and other malignancy or disorders.

Examples of an autoimmune disorder or disease include, but are not limited to, rheumatoid arthritis (RA), rheumatoid spondylitis, osteoarthritis and gouty arthritis, allergy, multiple sclerosis, autoimmune diabetes, autoimmune uveitis, and nephrotic syndrome.

Examples of an infectious disease include, but are not limited to, cachexia secondary to infection, acquired immune deficiency syndrome (AIDS) and AIDS-related complex (ARC), bacterial meningitis, cerebral malaria, cytomegalovirus infection secondary to transplantation, and fever and myalgias due to infection (such as influenza).

Examples of an intestinal disorder or disease include, but are not limited to, idiopathic inflammatory bowel disease, including Crohn's disease (adult of juvenile) and ulcerative colitis.

Examples of a pulmonary disorder or disease include, but are not limited to, adult respiratory distress syndrome, shock lung, chronic pulmonary inflammatory disease, pulmonary sarcoidosis, pulmonary fibrosis, and silicosis.

Examples of a cardiac disorder or disease include, but are not limited to, ischemia of the heart and hear insufficiency (weakness of the heart muscle).

Other malignancy or disorders under the definition of TNFα disorders include, but are not limited to, cachexia, cancer (including tumor growth or metastasis), inflammatory bone disorders and bone resorption disease, hepatitis (including alcoholic hepatitis and viral hepatitis), coagulation disturbances, perfusion injury, scar tissue formation, pyrexia, periodontal disease, obesity and radiation toxicity, adult Still's disease, Alzheimer's disease, ankylosing spondylitis, asthma, cancer and cachexia, atherosclerosis, chronic atherosclerosis, chronic fatigue syndrome, liver failure, chronic liver failure, obstructive pulmonary disease, chronic obstructive pulmonary disease, congestive heart failure, dermatopolymyositis, diabetic macrovasculopathy, endometriosis, familial periodic fevers, fibrosis, hemodialysis, Jarisch-Herxheimer reaction, juvenile rheumatoid arthritis, Kawasaki syndrome, myelo dysplastic syndrome, myocardial infarction, panciaticular vulgaris, periodontal disease, peripheral neuropathy, polyarticular, polymyositis, progressive renal failure, psoriasis, psoriatic arthritis, Reiter's syndrome, sarcoidosis, scleroderma, spondyloarthropathies, Still's disease, stroke, therapy associated syndrome, therapy induced inflammatory syndrome, inflammatory syndrome following TNFα administration, thoracoabdominal aortic aneurysm repair (TAAA), Vasulo-Behcet's disease, Yellow Fever vaccination, type 1 diabetes mellitus, type 2 diabetes mellitus, neuropathic pain, sciatica, cerebral edema, edema in and/or around the spinal cord, vasculitide, Wegener's granulomatosis, temporal arteritis, polymyalgia rheumatica, Takayasu's arteritis, polyarteritis nodosa, microscopic polyangiitis, Churg-Strauss syndrome, Felty's syndrome, Sjogren's syndrome, mixed connective tissue disorder, relapsing polychondritis, pseudogout, loosening of prostheses, autoimmune hepatitis, sclerosing cholangitis, acute pancreatitis, chronic pancreatitis, glomerulonephritides, post-streptococcal glomerulonephritis or IgA nephropathy, rheumatic heart disease, cardiomyopathy, orchitis, pyoderma gangrenosum, multiple myeloma, TNF receptor associated periodic syndrome [TRAPS], atherosclerosis, steroid dependent giant cell arteritismyostitis, uveitis, drug reactions, lupus, axial spondyloarthritis, axial spondyloarthritis without radiographic evidence of ankylosing spondylitis, pediatric Crohn's disease, psoriasis arthropathica, intestinal Behcet's disease, or hidradenitis suppurativa.

Accordingly, in some embodiments, provided is a method of treating or inhibiting a TNFα related disorder in a subject in need thereof comprising administering to the subject a therapeutically effective amount of the formulation as described herein, wherein the TNFα related disorder is selected from the group consisting of rheumatoid arthritis, juvenile idiopathic arthritis, axial spondyloarthritis, psoriatic arthritis, ankylosing spondylitis, axial spondyloarthritis without radiographic evidence of ankylosing spondylitis, Crohn's disease (e.g., adult), pediatric Crohn's disease, ulcerative colitis, psoriasis arthropathica, intestinal behcet's disease, plaque psoriasis, and hidradenitis suppurativa.

In some embodiments, the formulation of the present invention can be administered directly into the blood stream, into muscle, into tissue, into fat, or into an internal organ of a subject. Suitable means for parenteral administration include intravenous, intraarterial, intraperitoneal, intrathecal, intraventricular, intraurethral, intrasternal, intracranial, intramuscular, intra-ossial, intradermal and subcutaneous. Suitable devices for parenteral administration include needle (including microneedle, microprojections, soluble needles and other micropore formation techniques) injectors, needle-free injectors and infusion techniques. In some embodiments, the formulation of the present invention is administered to the subject subcutaneously.

In some embodiments, the administration pattern of the formulation of the present invention comprises administration of a dose of the formulation once every week, once every two weeks, once every three weeks, once every four weeks, once every five weeks, once every six weeks, once every seven weeks, once every eight weeks, once every nine weeks, once every ten weeks, once every fifteen weeks, once every twenty weeks, once every twenty five weeks, or once every twenty six weeks. In some embodiments, the formulation of the present invention is administered once every month, once every two months, once every three months, once every four months, once every five months, or once every six months. In some embodiments, the administration pattern of the medicament comprises administration of a dose of the formulation once every four or eight weeks.

In some embodiments the volume of a dose in the formulation is less than or equal to about 20 ml, about 15 ml, about 10 ml, about 5 ml, about 2.5 ml, about 1.5 ml, about 1.0 ml, about 0.75 ml, about 0.5 ml, about 0.25 ml or about 0.01 ml.

In some embodiments, the volume of a dose in the formulation is about 20 ml, about 19 ml, about 18 ml, about 17 ml, about 16 ml, about 15 ml, about 14 ml, about 13 ml, about 12 ml, about 11 ml, about 10 ml, about 9 ml, about 8 ml, about 7 ml, about 6 ml, about 5 ml, about 4 ml, about 3 ml, about 2 ml or about 1ml. Alternatively about 20.5 ml, about 19.5 ml, about 18.5 ml, about 17.5 ml, about 16.5 ml, about 15.5 ml, about 14.5 ml, about 13.5 ml, about 12.5 ml, about 11.5 ml, about 10.5 ml, about 9.5 ml, about 8.5 ml, about 7.5 ml, about 6.5 ml, about 5.5 ml, about 4.5 ml, about 3.5 ml, about 2.5 ml, about 1.5 ml, or about 0.5. Alternatively about 900 microliters, about 800 microliters, about 700 microliters, about 600 microliters, about 500 microliters, about 400 microliters, about 300 microliters, about 200 microliters, or about 100 microliters, alternatively about 950 microliters, about 850 microliters, about 750 microliters, about 650 microliters, about 550 microliters, about 450 microliters, about 350 microliters, about 250 microliters, about 150 microliters, or about 50 microliters. In some embodiments, the volume of the dose in a formulation is about 1.0 ml.

According to a preferred embodiment, the dose of the antibody in the formulation is between about 1-150 mg, about 5-145 mg, about 5-80 mg, about 10-140 mg, about 15-135 mg, about 20-130 mg, about 25-125 mg, about 25-50 mg, about 30-120 mg, about 35-115 mg, about 40-110 mg, about 45-105 mg, about 50-100 mg, about 55-95 mg, about 60-90 mg, about 65-85 mg, about 70-80 mg, or about 75 mg. For example, in some embodiments, the dose of the antibody in the formulation contains less than or equals to about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56 mg, about 57 mg, about 58 mg, about 59 mg, about 60 mg, about 70 mg, about 75 mg, about 80 mg, about 90 mg, about 100 mg, about 110 mg, about 120 mg, about 130 mg, about 140 mg, or about 150 mg. In some embodiments, the antibody is adalimumab (e.g., HUMIRA® or D2E7) in the dosing amount of 40 mg.

Dosage regimens may depend on the pattern of pharmacokinetic decay that the practitioner wishes to achieve. For example, in some embodiments, dosing from one-four times a week is contemplated. Even less frequent dosing may be used. In some embodiments, the dose is administered once every 1 week, every 2 weeks, every 3 weeks, every 4 weeks, every 5 weeks, every 6 weeks, every 7 weeks, every 8 weeks, every 9 weeks, every 10 weeks, every 15 weeks, every 20 weeks, every 25 weeks, or longer. In some embodiments, the dose is administered once every 1 month, every 2 months, every 3 months, every 4 months, every 5 months, every 6 months, or longer. The progress of this therapy is easily monitored by conventional techniques and assays. The dosing regimen can vary over time. For example, in some embodiments, the dose of the formulation of the present invention is administered at 40 mg every other week (e.g., by subcutaneous injection). In some embodiments, the dose of the formulation of the present invention is administered 20 mg every other week (e.g., by subcutaneous injection). In some embodiments, the dose of the formulation of the present invention is administered at an initial dose of four 40 mg subcutaneous injections in one day (at day 1) or two 40 mg injections per day for two consecutive days (at days 1 and 2), followed by a second dose two weeks later (at day 15) at 80 mg, and followed by a maintenance dose of 40 mg every other week. In some embodiments, the dose of the formulation of the present invention is administered at an initial dose of 80 mg (e.g., subcutaneous injection), followed by 40 mg every other week starting one week after initial dose.

For the purpose of the present invention, the appropriate dosage of the medicament will depend on the antibody employed, the type and severity of the disorder to be treated, whether the agent is administered for preventative or therapeutic purposes, previous therapy, the patient's clinical history and response to the agent, and the discretion of the attending physician. Typically the clinician will administer the medicament, until a dosage is reached that achieves the desired result. Dosages may be determined empirically. For example individuals are given incremental dosages to assess efficacy of the medicament, blood glucose levels may be followed.

Dose and/or frequency can vary over course of treatment. Empirical considerations, such as the antibody half-life, generally will contribute to the determination of the dosage. Frequency of administration may be determined and adjusted over the course of therapy, and is generally, but not necessarily, based on treatment and/or suppression and/or amelioration and/or delay of one or more symptoms of autoimmune disease. In some individuals, more than one dose may be required. Frequency of administration may be determined and adjusted over the course of therapy. For example without limitation, for repeated administrations over several days or longer, depending on the disease and its severity, the treatment is sustained until a desired suppression of symptoms occurs or until sufficient therapeutic levels are achieved to reduce blood glucose levels.

Administration of the formulation of the present invention can be continuous or intermittent, depending, for example, upon the recipient's physiological condition, whether the purpose of the administration is therapeutic or prophylactic, and other factors known to skilled practitioners. The administration of the formulation of the present invention can be essentially continuous over a preselected period of time or may be in a series of spaced dose.

Preferably the administration of the dose is a parenteral administration preferably selected from intravenous, intraarterial, intraperitoneal, intrathecal, intraventricular, intraurethral, intrasternal, intracranial, intramuscular, intra-ossial, intradermal and subcutaneous. Preferably the formulation is in a unit dosage sterile form for parenteral administration (e.g., subcutaneous administration).

The following examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Stability Study of the Adalimumab-PF Antibody Formulation

To evaluate the adalimumab-PF formulation of choice, a stability study including adalimumab in the PF formulation (see Table 1) and adalimumab in the commercial formulation (i.e., HUMIRA®) was performed. Adalimumab-PF drug substance from a developmental batch was formulated to prepare the adalimumab in the PF formulation and adalimumab in the commercial formulation (i.e., 50 mg/mL adalimumab, 12 mg/mL mannitol, 0.86 mg/mL monobasic sodium phosphate dehydrate, 1.52 mg/mL dibasic sodium phosphate dehydrate, 0.30 mg/mL sodium citrate, 1.3 mg/mL citric acid monohydrate, 6.16 mg/mL sodium chloride, 1 mg/mL polysorbate 80, pH 5.2). The data were also compared with that from the stability study on a representative lot of the adalimumab licensed product (EU lot 87010XD06) and presented together in the figures to provide a preliminary assessment of similarity between formulations.

TABLE 1

| Name of Ingredients | Unit Formula (mg/mL) |
|---|---|
| Adalimumab | 50 |
| Histidine (L-histidine) | 0.786 |
| L-histidine hydrochloride monohydrate | 3.132 |
| Sucrose | 85 |
| Disodium edetate dihydrate (EDTA) | 0.05 |
| L-methionine | 0.2 |
| Polysorbate 80 (Crillet 4 HP) | 0.2 |
| Water for Injection | q.s. to 1 mL |

The adalimumab samples were filled with 0.8 mL of drug product in 1 mL staked-in needle syringes with latex-free rigid needle shield, stoppered with fluoropolymer coated plungers and stored horizontally at 2-8° C., 25° C., and 40° C. The adalimumab licensed product was enrolled within the studies in the commercial presentation, without manipulation. Samples were then analyzed for quality attributes that are commonly used to monitor product degradation using SE-HPLC, iCE, and rCGE. Oxidation was also assessed by measuring % oxidation of Met-253.

Data were collected for 6 months at 2-8° C. and 25° C., and for 3 months at 40° C. No significant changes were observed at 2-8° C.

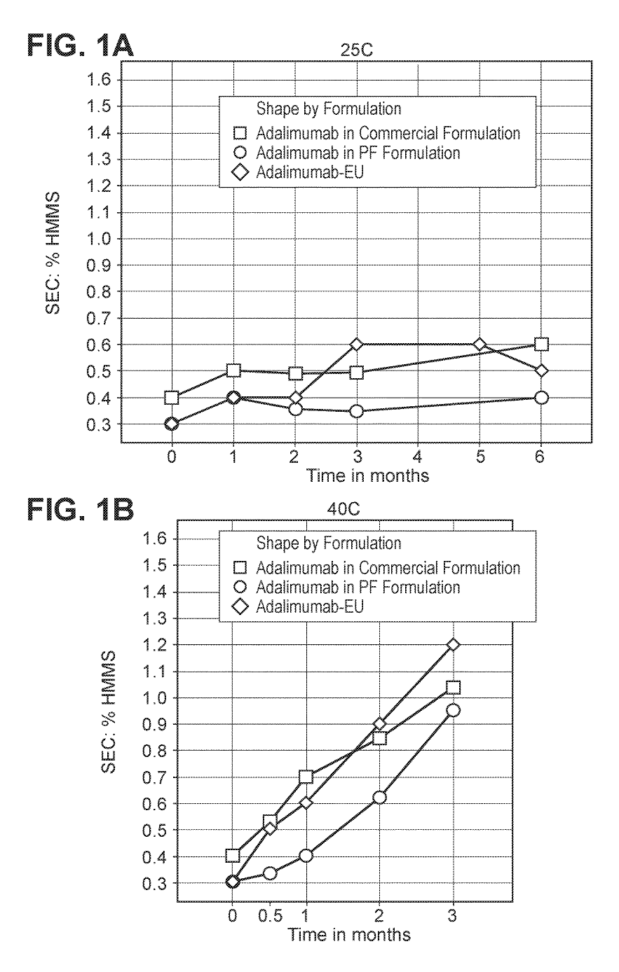

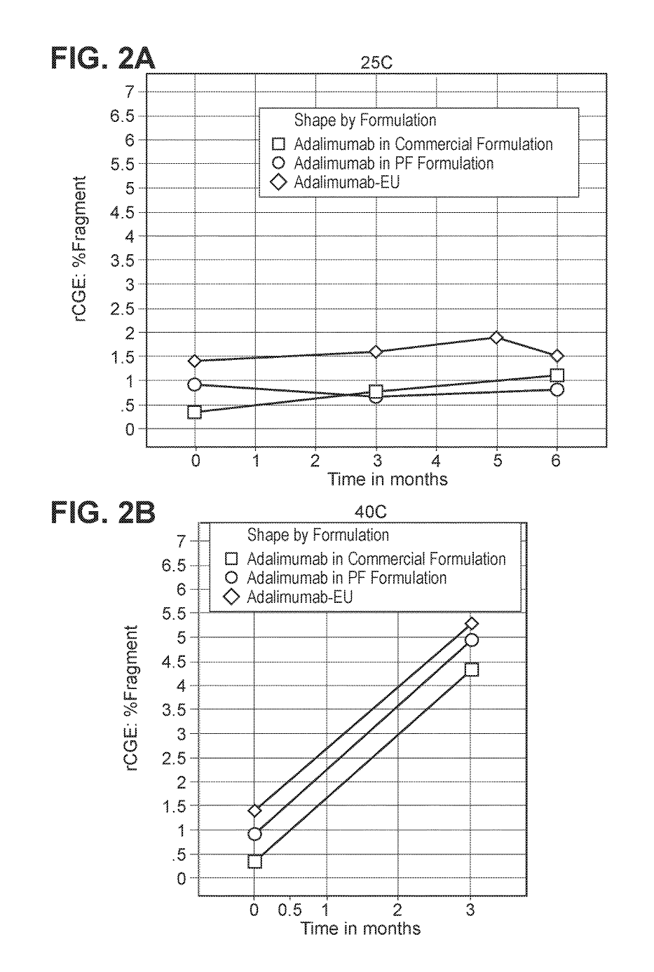

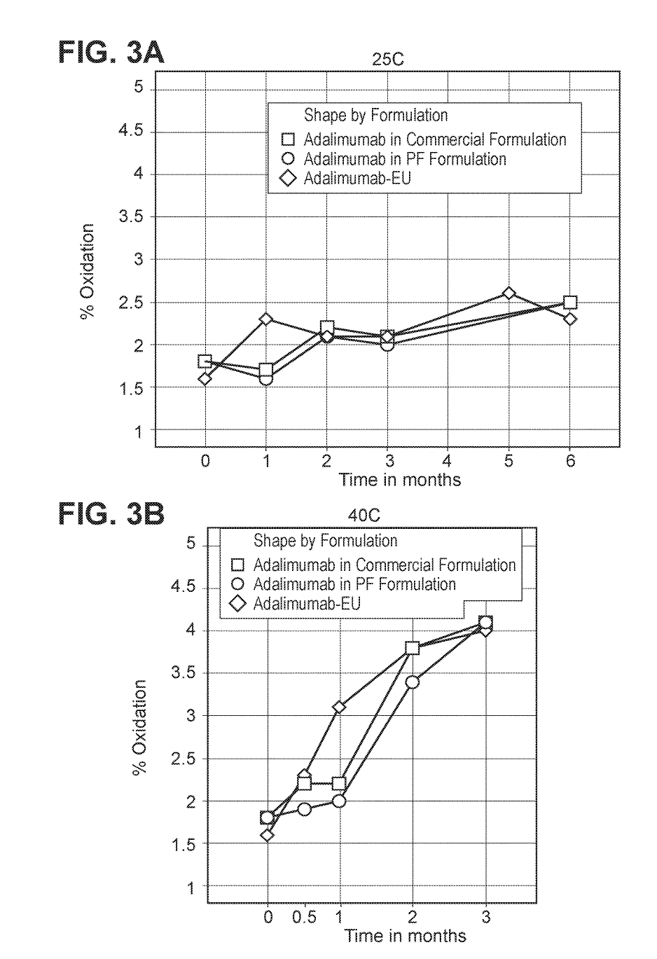

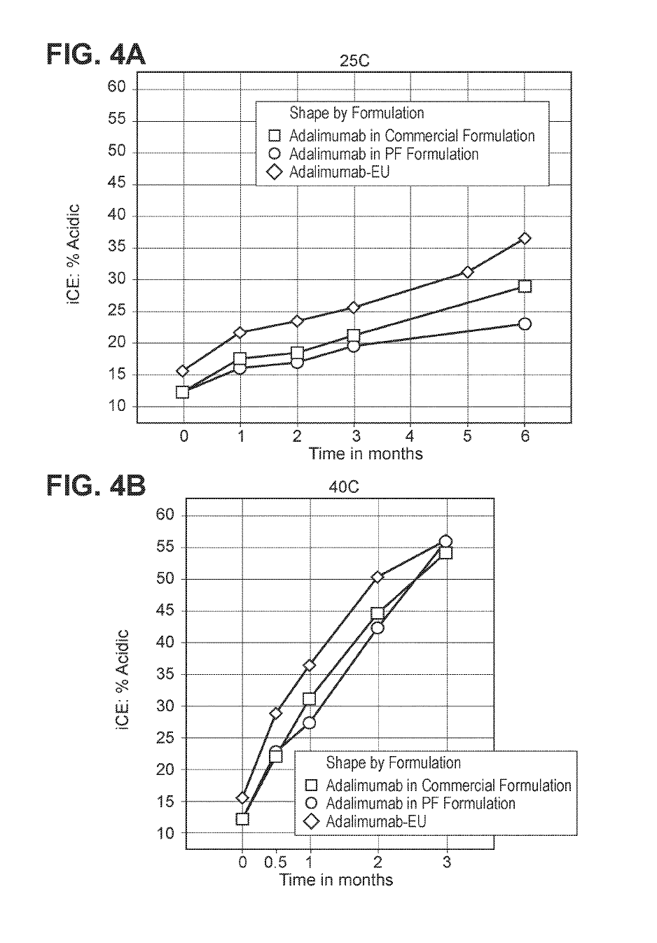

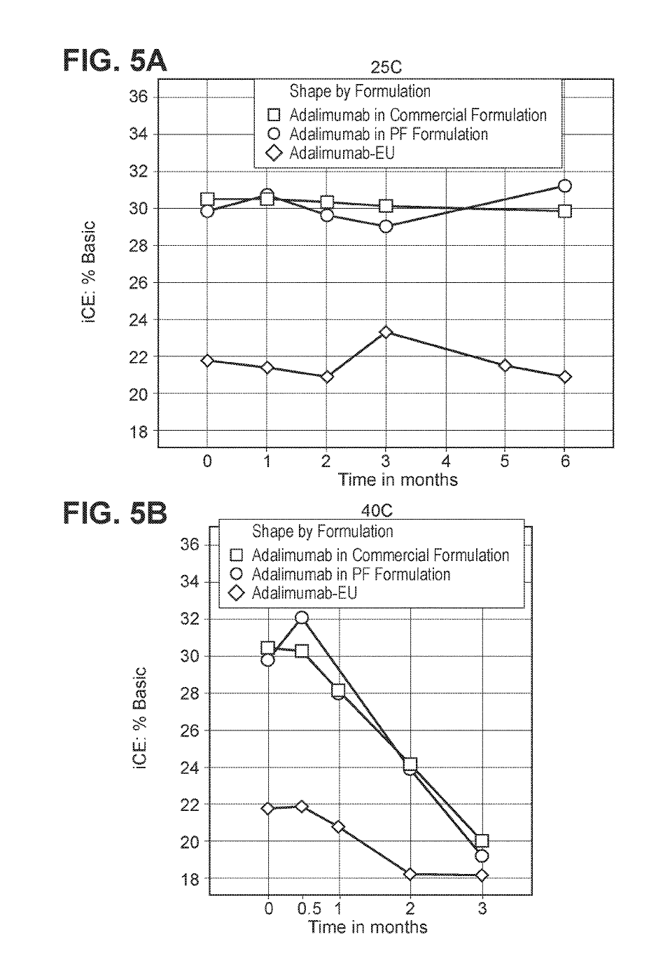

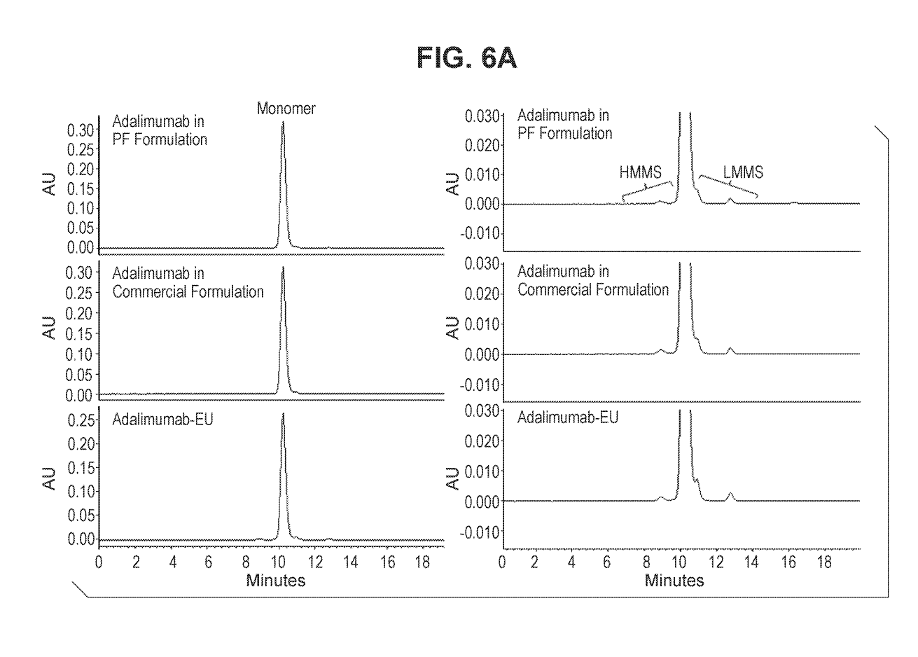

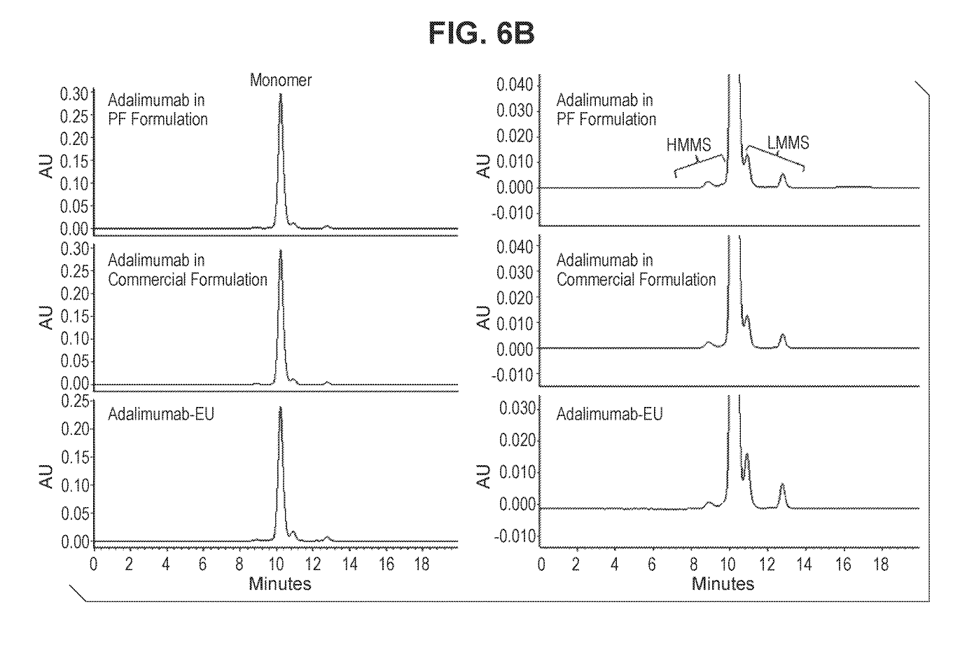

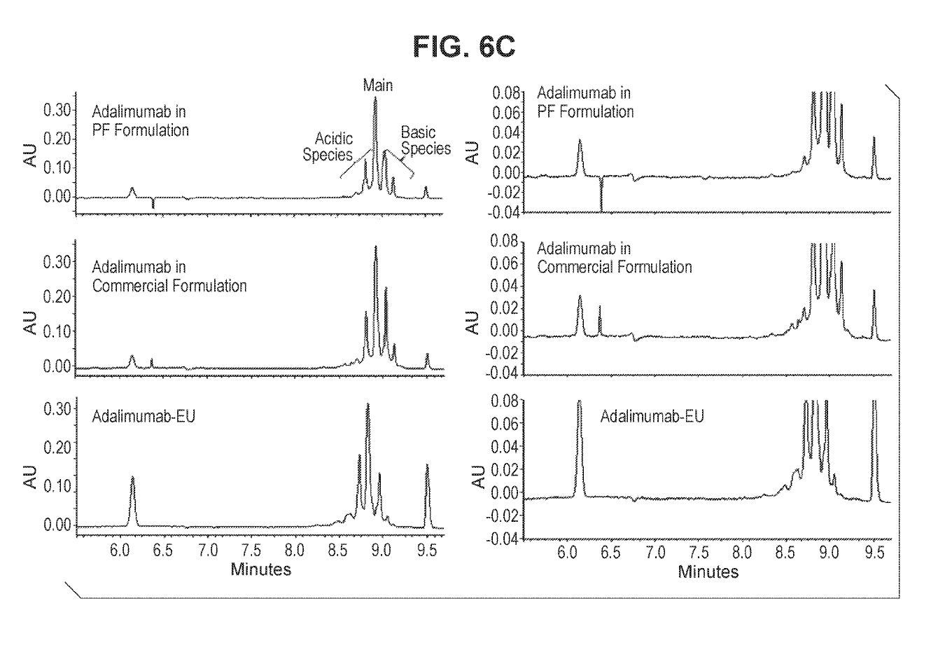

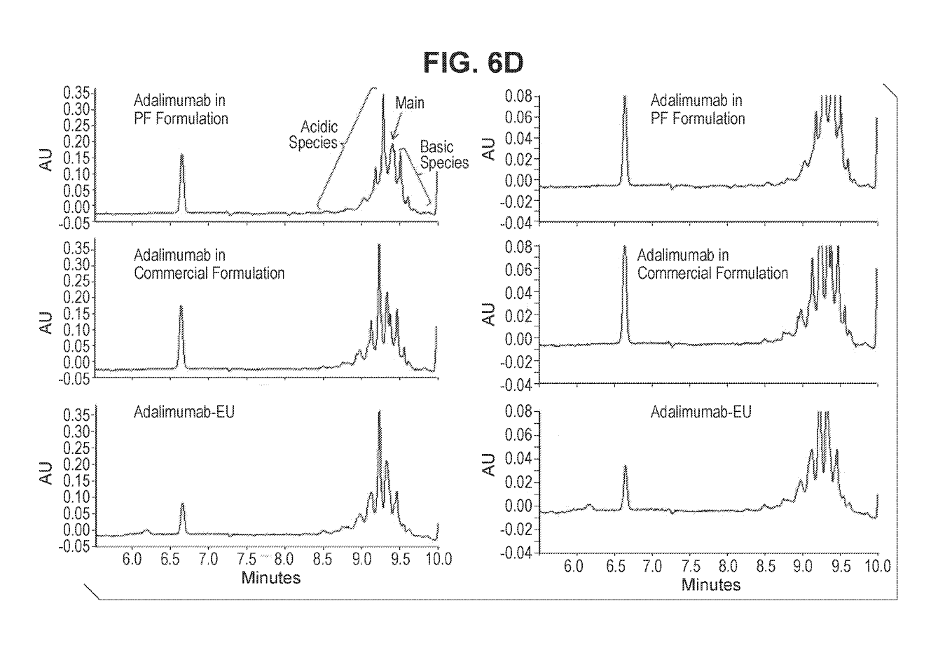

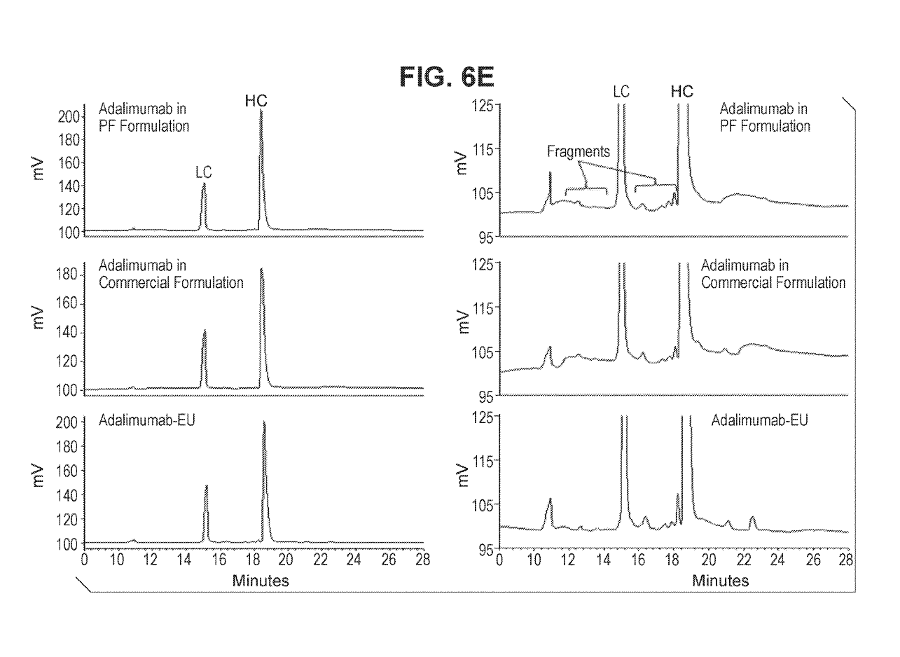

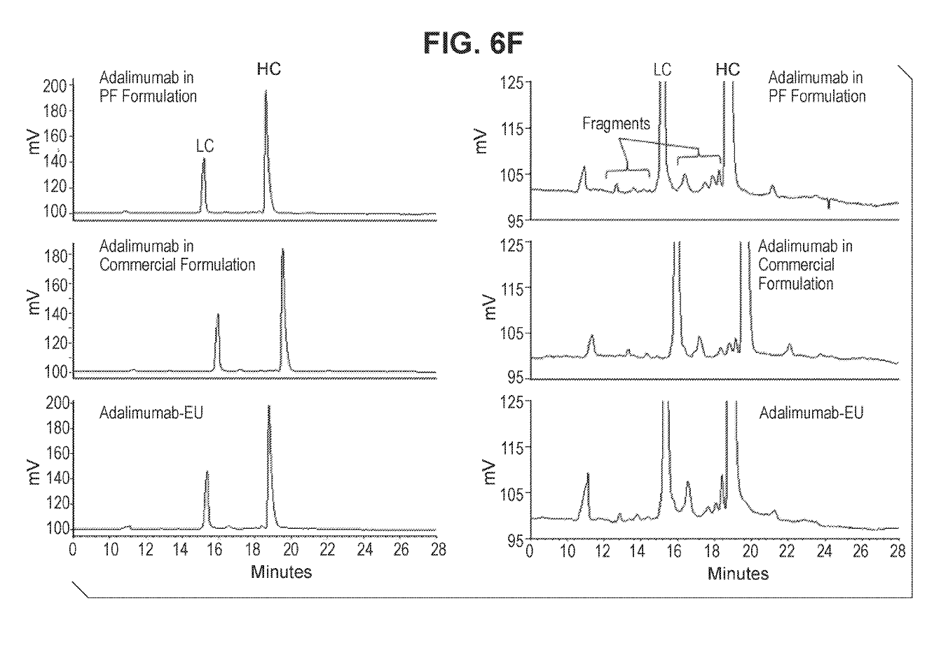

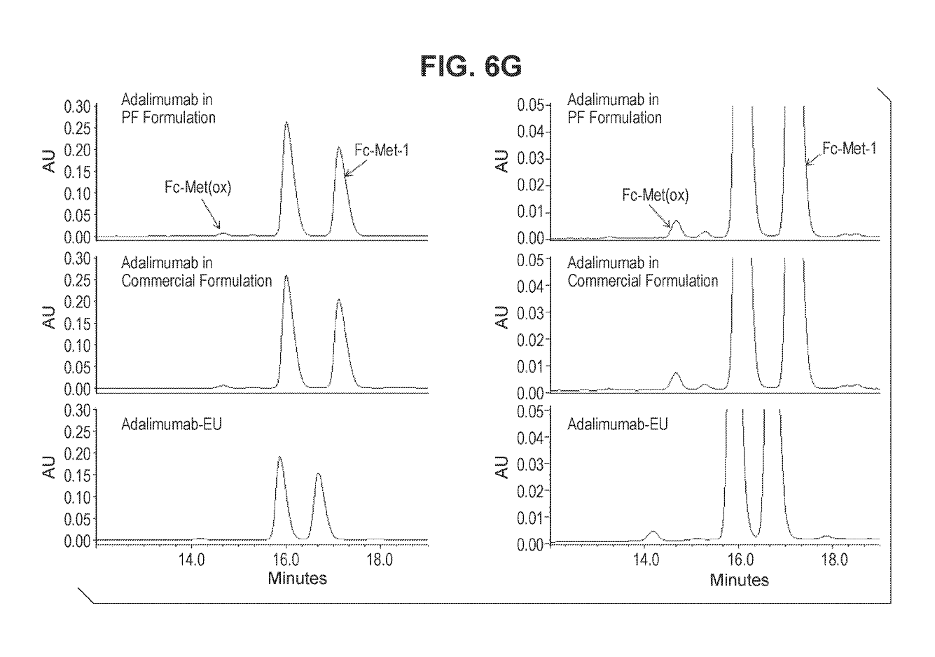

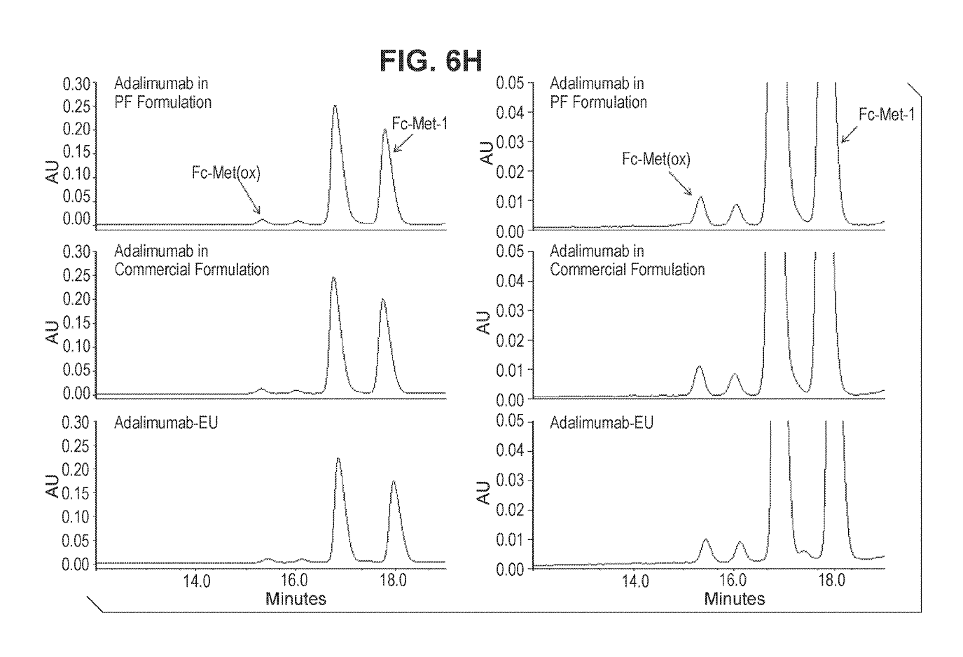

For high molecular mass species (% HMMS; FIGS. 1A and 1B), % fragmentation by rCGE (FIGS. 2A and 2B) and Met-253 oxidation data (FIGS. 3A and 3B) at 25° C. and 40° C., the degradation profiles were similar between adalimumab in the PF formulation, adalimumab in the commercial formulation, and adalimumab licensed product. For acidic species data at 25° C. (FIG. 4A), the adalimumab in the PF formulation showed less change in acidic species after 6 months compared to the other two formulations. At 40° C. after 3 months (FIG. 4B), the charged species profiles showed similar change in acidic species formed for all three formulations. For basic species, time zero levels for adalimumab-PF formulation was higher than the commercial formulation. No apparent change in basic species was observed for all three formulations at 25° C. (FIG. 5A). At 40° C. (FIG. 5B), a decrease in basic species was observed for all three formulations. Adalimumab in the PF formulation and adalimumab in the commercial formulation was more prominent than observed in the adalimumab licensed product. Further, comparison of SE-HPLC, iCE, rCGE, and Met-253 oxidation after storage at 25° C. for 6 months and 40° C. for 3 months show that no new degradation species were observed for the adalimumab product that were not also observed in the adalimumab licensed products, and the overall stability performance of the adalimumab product was similar to the adalimumab licensed products under the same conditions (See FIGS. 6A-6H).

Overall, adalimumab in the PF formulation showed similar stability to adalimumab in the commercial formulation and the adalimumab licensed product following storage at elevated temperatures (25° C. and 40° C.). Additional studies, such as subjecting samples to agitation stress, demonstrated no apparent differences for adalimumab product formulated in either the PF or commercial formulation. Further, the data demonstrated that the PF formulation is capable of conferring satisfactory stability to adalimumab and that adalimumab in this formulation performed similarly to the adalimumab licensed product. No new degradation products were observed for samples using the adalimumab-PF formulation as compared to the adalimumab in commercial formulation and the licensed product, and the overall degradation profiles appeared similar.

### Example 2

### Comparative Forced Degradation Study of the Adalimumab-PF Drug Product and Adalimumab-US/EU at Elevated Temperature

Full-scale adalimumab-PF drug product (3 lots) was enrolled in the elevated temperature (forced degradation) study with storage at 40° C. for three months. A total of 3 lots of adalimumab-US (i.e., reference product HUMIRA® in US) and 3 lots of adalimumab-EU (i.e., reference product HUMIRA® in EU) drug product (1 previous and 2 current versions) were also enrolled in the same elevated temperature study, as summarized in Table 2.

TABLE 2

| Analytical | | Utility in Assessment of | Results and Conclusions |
|---|---|---|---|
| Procedure | Attribute | Similarity | (Summary)^{a} |
| Appearance | Coloration, | Qualitative comparison | Adalimumab-PF |
| pH | clarity and | For information only | formulations were less |
| UV | visual particles | (similarity not required) | opalescent than reference |
| Spectroscopy | pH | | product; all samples were |
| | | | essentially free of visible |
| | | | particles, were generally |
| | | | clear to very slightly brown. |
| | | | No change in pH |
| | | | observed. |
| | Protein | If change was observed, | No change in protein |
| | concentration | quantitative assessment | concentration observed. |
| | | of change | |
| Continuation | Adalimumab- | Compared full-scale | Assessment of similarity |
| | PF, 40 mg | adalimumab-PF to | |
| | PFS (Ph1 | reference products to | |
| | syringes) | assess similarity | |
| | 3 full-scale lots | Compared reference | |
| | (3 GMP) | products to one | |
| | | another to assess | |
| | | similarity of | |
| | | adalimumab-US to | |
| | | adalimumab-EU | |

The same degradation products were observed for adalimumab-PF, adalimumab-US and adalimumab-EU samples (40 mg PFS), suggesting a similar degradation profile. For this study, adalimumab-US and adalimumab-EU products were procured and placed on stability at 40° C. along with the GMP (Good Manufacturing Practice) adalimumab-PF product (Phi PFS). The lots enrolled within the elevated temperature study were selected based on availability, and were within their registered expiry date. After three months of storage at 40° C., samples were analyzed for degradation. A summary of the analytical methods used and results of the analytical assessment for the elevated temperature study is shown in Table 3A, and Tables 3B-3D summarize the quantitative data for integrated charged variants after one month storage at 40° C.

TABLE 3A

| Analytical | | Utility in Assessment of | Results and Conclusions |
|---|---|---|---|
| Procedure | Attribute | Similarity | (Summary) |
| Appearance | Coloration, | Qualitative comparison | Adalimumab-PF |
| | clarity and | | formulations were less |
| | visual particles | | opalescent than reference |
| | | | product; all samples were |
| | | | essentially free of visible |
| | | | particles, were generally |
| | | | clear to very slightly brown. |
| pH | pH | For information only | No change in pH observed. |
| | | (similarity not required) | |
| UV | Protein | If change was observed, | No change in protein |
| Spectroscopy | concentration | quantitative assessment | concentration observed. |
| | | of change | |
| SE-HPLC | HMMS, | Qualitative assessment of | All samples had an |
| | LMMS | chromatographic profiles, | increase in HMMS with |
| | | quantitative comparison of | adalimumab-PF showing |
| | | change in amounts of | lesser rate of formation; |
| | | HMMS and LMMS | relatively higher proportion |
| | | | observed for LMMS for all |
| | | | products |
| iCE | Molecular | Qualitative assessment of | All samples showed |
| | Charge | electropherogram profiles, | increases in the relative |
| | | quantitative assessment | proportion of acidic species |
| | | of relative changes in | and decreases in the |
| | | acidic (%), basic (%), and | relative proportion of |
| | | main (%) species | predominantly main |
| | | | species. |
| CGE | H and L | Qualitative assessment of | All samples had |
| (Reducing) | integrity, | electropherogram profiles, | comparable increase in |
| | Fragments | quantitative assessment | fragment. |
| | | of relative change in | |
| | | fragments | |
| CGE (non- | Intact IgG | Qualitative assessment of | All samples had |
| reducing) | Fragment | electropherogram profiles, | comparable increase in |
| | | quantitative assessment | fragment. |
| | | of relative change in | |
| | | fragments | |
| Cell-Based | Relative | Quantitative assessment | On average, all samples |
| Bioassay | Potency | of potency | showed a slight decrease in |
| | | | relative potency. |
| HIAC^{a} | Sub-visible | Semi-quantitative | Adalimumab-PF showed |
| | particulates | assessment of sub-visible | low sub-visible counts at |
| | | particle levels | T0. All samples showed an |
| | | | increase in sub-visible |
| | | | particulate matter and |
| | | | significant variability among |
| | | | samples after storage at |
| | | | 40° C. for three months. |

Abbreviations:
UV = Ultraviolet;
iCE = Imaged Capillary Electrophoresis,
CGE = Capillary Gel Electrophoresis,
HMMS = High Molecular Mass Species,
LMMS = Low Molecular Mass Species
H = Heavy Chain,
L = Light Chain.
^{a}HIAC method is a modified method run in the development laboratory.

TABLE 3B
Summary Table of Forced Degradation of Adalimumab-PF Lots stored at 40° C.

| Source and Lot | | adalimumab-PF | | | adalimumab-PF | | | adalimumab-PF | | |
|---|---|---|---|---|---|---|---|---|---|---|
| Number | | Lot 008A13 | | | Lot 003C13 | | | Lot 004C13 | | |
| Syringe Type | | Ph1 | | | Ph1 | | | Ph1 | | |
| Analytical | | Value at | Value at | | Value at | Value at | | Value at | Value at | |
| Procedure | Parameter | T0 | 3 Months | | T0 | 3 Month | | T0 | 3 Months | |
| Appearance | Visible | EFVP | EFVP | | EFVP | EFVP | | EFVP | EFVP | |
| | Particles | | | | | | | | | |
| | Clarity | NMOPS I | NMOPS I | | NMOPS I | NMOPS I | | NMOPS I | NMOPS I | |
| | Color | B7 | B7 | | B7 | B7 | | B7 | B7 | |
| Analytical | | Value at | Value at | Change | Value at | Value at | Change | Value at | Value at | Change |
| Procedure | Parameter | T0 | 3 Months | from T0 | T0 | 3 Months | from T0 | T0 | 3 Months | from T0 |
| pH | pH | 5.6 | 5.5 | −0.1 | 5.6 | 5.5 | −0.1 | 5.6 | 5.5 | −0.1 |
| UV | Protein | 48.5 | 48.5 | 0.0 | 48.1 | 47.0 | −1.1 | 51.5 | 51.3 | −0.2 |
| | Concentra- | | | | | | | | | |
| | tion | | | | | | | | | |
| | (mg/mL) | | | | | | | | | |
| Bioassay | Relative | 102 | 85 | −17 | 107 | 82 | −25 | 99 | 85 | −14 |
| | Potency (%) | | | | | | | | | |
| HIAC | ≧10 μm | 121 | 2707 | 2586 | 127 | 1434 | 1307 | 80 | 1986* | 1906 |
| Sub- | per mL | | | | | | | | | |
| visible | ≧25 μm | 3 | 176 | 173 | 5 | 131 | 126 | 3 | 76* | 73 |
| Particles | per ML | | | | | | | | | |
| Size | % Monomer | 99.5 | 93.3 | −6.2 | 99.5 | 93.1 | −6.4 | 99.5 | 93.0 | −6.5 |
| Exclusion | % HMMS | 0.4 | 1.0 | 0.6 | 0.4 | 1.0 | 0.6 | 0.4 | 0.9 | 0.5 |
| HPLC | % LMMS | 0.1 | 5.7 | 5.6 | 0.1 | 5.9 | 5.8 | 0.1 | 6.1 | 6.0 |
| CGE- | Heavy | 100.0 | 95.1 | −4.9 | 100.0 | 95.0 | −5.0 | 100.0 | 95.0 | −5.0 |
| Reduced | Chain + Light | | | | | | | | | |
| | Chain (%) | | | | | | | | | |
| | % Fragment | 0.0 | 4.3 | 4.3 | 0.0 | 4.0 | 4.0 | 0.0 | 4.1 | 4.1 |
| | % Other | 0.0 | 0.6 | 0.6 | 0.0 | 1.0 | 1.0 | 0.0 | 0.9 | 0.9 |
| CGE- | % mAb | 97.3 | 88.4 | −8.9 | 97.8 | 88.3 | −9.5 | 98.0 | 89.6 | −8.4 |
| Non- | % Fragment | 2.7 | 11.6 | 8.9 | 2.2 | 11.7 | 9.5 | 2.0 | 10.4 | 8.4 |
| Reduced | % Other | 0.0 | 0.0 | 0.0 | 0.0 | 0.0 | 0.0 | 0.0 | 0.0 | 0.0 |
| iCE | Acidic | 24.3 | 65.2 | 40.9 | 26.2 | 70.0 | 43.8 | 25.7 | 67.8 | 42.1 |
| | Peaks (%) | | | | | | | | | |
| | Main | 53.8 | 14.4 | −39.4 | 59.8 | 15.0 | −44.8 | 56.3 | 15.1 | −41.2 |
| | Peaks (%) | | | | | | | | | |
| | Basic | 21.9 | 20.4 | −1.5 | 14.0 | 15.0 | 1.0 | 18.1 | 17.1 | −1.0 |
| | Peaks (%) | | | | | | | | | |

EFVP = Essentially Free Of Visible Particles,
NMOPS = Not More Opalescent than Standard,
*= HIAC Sensor Limit Exceeded

TABLE 3C
Summary Table of Forced Degradation of Adalimumab-US Lots stored at 40° C.

| Source and Lot | | Reference Product-US | | | Reference Product-US | | | Reference Product-US | | |
|---|---|---|---|---|---|---|---|---|---|---|
| Number | | Lot 240482E | | | Lot 260972E | | | Lot 260982E | | |
| Syringe Version | | Current | | | Current | | | Current | | |
| Analytical | | Value at | Value at | | Value at | Value at | | Value at | Value at | |
| Procedure | Parameter | T0 | 3 Months | | T0 | 3 Month | | T0 | 3 Months | |
| Appearance | Visible | EFVP | EFVP | | EFVP | EFVP | | EFVP | EFVP | |
| | Particles | | | | | | | | | |
| | Clarity | NMOPS III | NMOPS III | | NMOPS III | NMOPS III | | NMOPS III | NMOPS III | |
| | Color | B7 | B7 | | B7 | B7 | | B7 | B7 | |
| Analytical | | Value at | Value at | Change | Value at | Value at | Change | Value at | Value at | Change |
| Procedure | Parameter | T0 | 3 Months | from T0 | T0 | 3 Months | from T0 | T0 | 3 Months | from T0 |
| pH | pH | 5.4 | 5.3 | −0.1 | 5.4 | 5.3 | −0.1 | 5.4 | 5.3 | −0.1 |
| UV | Protein | 48.3 | 47.8 | −0.5 | 47.4 | 46.6 | −0.8 | 48.6 | 48.1 | −0.5 |
| | Concentra- | | | | | | | | | |
| | tion | | | | | | | | | |
| | (mg/mL) | | | | | | | | | |
| Bioassay | Relative | 98 | 86 | −12 | 95 | 86 | −9 | 106 | 82 | −24 |
| | Potency (%) | | | | | | | | | |
| HIAC | ≧10 μm | 1324 | 1883* | 559 | 1455 | 1322 | −133 | 2693 | 1958 | −735 |
| Sub- | per mL | | | | | | | | | |
| visible | ≧25 μm | 40 | 68* | 28 | 45 | 44 | −1 | 63 | 51 | −12 |
| Particles | per ML | | | | | | | | | |
| Size | % Monomer | 99.6 | 86.9 | −12.7 | 99.6 | 87.8 | −11.8 | 99.6 | 89.6 | −10.0 |
| Exclusion | % HMMS | 0.3 | 4.4 | 4.1 | 0.3 | 3.4 | 3.1 | 0.3 | 2.7 | 2.4 |
| HPLC | % LMMS | 0.1 | 8.8 | 8.7 | 0.1 | 8.8 | 8.7 | 0.1 | 7.7 | 7.6 |
| CGE- | Heavy | 99.0 | 93.6 | −5.4 | 99.1 | 93.7 | −5.4 | 99.0 | 93.9 | −5.1 |
| Reduced | Chain + Light | | | | | | | | | |
| | Chain (%) | | | | | | | | | |
| | % Fragment | 1.0 | 5.6 | 4.6 | 0.9 | 5.5 | 4.6 | 1.0 | 5.2 | 4.2 |
| | % Other | 0.0 | 0.8 | 0.8 | 0.0 | 0.9 | 0.9 | 0.0 | 0.9 | 0.9 |
| CGE- | % mAb | 98.0 | 84.0 | −14.0 | 98.1 | 84.4 | −13.7 | 98.1 | 86.2 | −11.9 |
| Non- | % Fragment | 2.0 | 16.0 | 14.0 | 1.9 | 15.6 | 13.7 | 1.9 | 13.8 | 11.9 |
| Reduced | % Other | 0.0 | 0.0 | 0.0 | 0.0 | 0.0 | 0.0 | 0.0 | 0.0 | 0.0 |
| iCE | Acidic | 21.7 | 59.4 | 37.7 | 21.6 | 60.4 | 38.8 | 21.3 | 60.2 | 38.9 |
| | Peaks (%) | | | | | | | | | |
| | Main | 51.8 | 15.7 | −36.1 | 53.0 | 16.3 | −36.7 | 52.2 | 15.6 | −36.6 |
| | Peaks (%) | | | | | | | | | |
| | Basic | 26.6 | 24.8 | −1.8 | 25.5 | 23.3 | −2.2 | 26.6 | 24.2 | −2.4 |
| | Peaks (%) | | | | | | | | | |

EFVP = Essentially Free Of Visible Particles,
NMOPS = Not More Opalescent than Standard,
*= HIAC Sensor Limit Exceeded

TABLE 3D
Summary Table of Forced Degradation of Adalimumab-EU Lots stored at 40° C.

| Source and Lot | | Reference Product-EU | | | Reference Product-EU | | | Reference Product-EU | | |
|---|---|---|---|---|---|---|---|---|---|---|
| Number | | Lot 13257XD10 | | | Lot 21327XH07 | | | Lot 25361XD01 | | |
| Syringe Version | | Previous | | | Current | | | Current | | |
| Analytical | | Value at | Value at | | Value at | Value at | | Value at | Value at | |
| Procedure | Parameter | T0 | 3 Months | | T0 | 3 Month | | T0 | 3 Months | |
| Appearance | Visible | EFVP | EFVP | | EFVP | EFVP | | EFVP | EFVP | |
| | Particles | | | | | | | | | |
| | Clarity | NMOPS III | NMOPS III | | NMOPS III | NMOPS III | | NMOPS III | NMOPS III | |
| | Color | B7 | B7 | | B7 | B7 | | B7 | B7 | |
| Analytical | | Value at | Value at | Change | Value at | Value at | Change | Value at | Value at | Change |
| Procedure | Parameter | T0 | 3 Months | from T0 | T0 | 3 Months | from T0 | T0 | 3 Months | from T0 |
| pH | pH | 5.4 | 5.3 | −0.1 | 5.3 | 5.3 | 0.0 | 5.4 | 5.3 | −0.1 |
| UV | Protein | 48.4 | 47.6 | −0.8 | 48.6 | 47.7 | −0.9 | 47.7 | 46.9 | −0.8 |
| | Concentra- | | | | | | | | | |
| | tion | | | | | | | | | |
| | (mg/mL) | | | | | | | | | |
| Bioassay | Relative | 105 | 80 | −25 | 108 | 83 | −25 | 106 | 83 | −23 |
| | Potency (%) | | | | | | | | | |
| HIAC | ≧10 μm | 1568.0 | 1338* | −230 | 729 | 1181* | 452 | 1023 | 786 | −237 |
| Sub- | per mL | | | | | | | | | |
| visible | ≧25 μm | 30 | 44* | 14 | 16 | 26* | 10 | 14 | 16 | 2 |
| Particles | per ML | | | | | | | | | |
| Size | % Monomer | 99.5 | 88.8 | −10.7 | 99.6 | 89.7 | −9.9 | 99.6 | 87.9 | −11.7 |
| Exclusion | % HMMS | 0.4 | 2.4 | 2.0 | 0.3 | 2.3 | 2.0 | 0.3 | 2.9 | 2.6 |
| HPLC | % LMMS | 0.2 | 8.8 | 8.6 | 0.1 | 8.0 | 7.9 | 0.1 | 9.2 | 9.1 |
| CGE- | Heavy | 99.2 | 93.6 | −5.6 | 99.2 | 94.2 | −5.0 | 99.3 | 93.9 | −5.4 |
| Reduced | Chain + Light | | | | | | | | | |
| | Chain (%) | | | | | | | | | |
| | % Fragment | 0.8 | 5.5 | 4.7 | 0.8 | 5.4 | 4.6 | 0.7 | 5.3 | 4.6 |
| | % Other | 0.0 | 0.9 | 0.9 | 0.0 | 0.4 | 0.4 | 0.0 | 0.8 | 0.8 |
| CGE- | % mAb | 97.6 | 84.3 | −13.3 | 97.8 | 85.6 | −12.2 | 97.7 | 83.8 | −13.9 |
| Non- | % Fragment | 2.4 | 15.7 | 13.3 | 2.2 | 14.4 | 12.2 | 2.3 | 16.2 | 13.9 |
| Reduced | % Other | 0.0 | 0.0 | 0.0 | 0.0 | 0.0 | 0.0 | 0.0 | 0.0 | 0.0 |
| iCE | Acidic | 23.1 | 62.3 | 39.2 | 21.8 | 61.6 | 39.8 | 23.8 | 63.9 | 40.1 |
| | Peaks (%) | | | | | | | | | |
| | Main | 55.6 | 17.2 | −38.4 | 53.8 | 15.9 | −37.9 | 57.1 | 16.2 | −40.9 |
| | Peaks (%) | | | | | | | | | |
| | Basic | 21.3 | 20.5 | −0.8 | 24.4 | 22.5 | −1.9 | 19.1 | 19.9 | 0.8 |
| | Peaks (%) | | | | | | | | | |

EFVP = Essentially Free Of Visible Particles,
NMOPS = Not More Opalescent than Standard,
*= HIAC Sensor Limit Exceeded

In summary, information collected from elevated temperature forced degradation conditions comparing the degradation profiles of adalimumab-US, adalimumab-EU, and adalimumab-PF show that adalimumab-PF is similar to adalimumab-US; adalimumab-EU is similar to adalimumab-US; adalimumab-PF is similar to adalimumab EU; and adalimumab-PF is similar to the pool of adalimumab commercial products (−US and −EU) irrespective of the container-closure used. Both the quantitative and qualitative results suggest that there are two predominant routes of degradation during storage at 40° C.: 1) Changes in the charge profile, demonstrated by increases in the relative amounts of acidic species and decreases in the relative amounts of predominantly main species as measured by iCE; and 2) increase of low molecular mass species as demonstrated by the increase in % fragment as measured by rCGE, nrCGE and increase in LMMS as measured by SE-HPLC. Observations during formulation development show that adalimumab was more stable in the PF formulation than the reference product formulation. These observations include lower levels of %HMMS, %LMMS and %fragment (nrCGE) measured in adalimumab-PF compared to adalimumab-US or adalimumab-EU.

In all instances the observed degradation species were the same for adalimumab-PF, adalimumab-US, and adalimumab-EU. No new degradation species were observed in the adalimumab-PF product that were not also observed in adalimumab-US and adalimumab-EU.

### Example 3

### Comparative Forced Degradation Study of the Adalimumab-PF Drug Product and Adalimumab-US/EU by Photodegradation

The objective of this study was to expose drug product to high intensity light conditions, analyze the resulting forcibly photo-degraded materials, and assess the similarity of the observed degradation pattern for adalimumab-PF materials as compared to adalimumab reference products.

The study utilized three lots of adalimumab-PF drug product (in Ph3 PFS), three lots of adalimumab-US drug product and three lots of adalimumab-EU drug product (current version). The latest version of container-closure for both the adalimumab-PF and adalimumab-EU product was used for the study. All materials in PFS (pre-filled syringe) were placed in a light chamber horizontally and exposed to approximately 8.0 klux of light for 7 days at a controlled temperature of 25° C. Table 4 lists the materials enrolled in the study; and the drug product materials were characterized at T=0 and T=7 days using the analytical techniques outlined in Table 5.

TABLE 4
Adalimumab Lots Enrolled in Forced Degradation
by Photoexposure Study
Adalimumab Reference Product Lots

| Source | Presentation | Lot # | Expiry |
|---|---|---|---|
| Adalimumab-US | 40 mg | 1010535 | May 2015 |
| Adalimumab-US | 40 mg | 1010534 | May 2015 |
| Adalimumab-US | 40 mg | 1010847 | May 2015 |
| Adalimumab-EU | 40 mg | 21362XH07 | August 2014 |
| (current version) | | | |
| Adalimumab-EU | 40 mg | 25365XH04 | December 2014 |
| (current version) | | | |
| Adalimumab-EU | 40 mg | 33425XD08 | August 2015 |
| (current version) | | | |

Adalimumab-PF Drug Product Lots

| | | | Date of |
|---|---|---|---|
| Source | Presentation | Lot # | Manufacture |
| Adalimumab-PF | 40 mg | 0211314 | April 2013 |
| (Ph3 PFS) | | | |
| Adalimumab-PF | 40 mg | 00706646-0045-B | April 2014 |
| (Ph3 PFS) | | | |
| Adalimumab-PF | 40 mg | 00706646-0045-C | April 2014 |
| (Ph3 PFS) | | | |

TABLE 5
Analytical Assessment for Comparative Forced Degradation Study

| | | Utility in | |
|---|---|---|---|
| Analytical | | Assessment of | Results and Conclusions |
| Procedure | Attribute | Similarity | (Summary) |
| Appearance | Coloration, | Qualitative | All samples were essentially free of |
| | clarity and | comparison | visible particles. No change from |
| | visual | | initial clarity. Change in color from |
| | particles | | B9 to B6. |
| pH | pH | For information only | No change from initial pH observed |
| | | (similarity not | for all samples. |
| | | required) | |
| UV | Protein | If change is | All samples had a protein |
| Spectroscopy | concentration | observed, | concentration of about 50 mg/mL. |
| | | quantitative | |
| | | assessment of | |
| | | change | |
| Methionine | Methionine | Qualitative | All samples showed increase in |
| oxidation | oxidation | assessment of | methionine oxidation levels. |
| | | chromatographic | Reference product showed higher |
| | | profiles, quantitative | oxidation. |
| | | comparison of | |
| | | change in amount of | |
| | | methionine oxidation. | |
| iCE | Molecular | Qualitative | All samples showed increases in |
| | Charge | assessment of | the relative proportion of acidic |
| | | electropherogram | species and decreases in the |
| | | profiles, quantitative | relative proportion of main and |
| | | assessment of | basic species. Adalimumab-Pfizer |
| | | relative changes in | showed higher acidic species |
| | | acidic (%), basic (%), | compared to reference drug |
| | | and main (%) | products. |
| | | species. | |
| SE-HPLC | HMMS | Qualitative | All samples had an increase in |
| | Monomer | assessment of | HMMS and a minor increase in |
| | LMMS | chromatographic | LMMS. Reference product showed |
| | | profiles, quantitative | higher increase in HMMS and |
| | | comparison of | LMMS. |
| | | change in amounts of | |
| | | HMMS, monomer | |
| | | and LMMS. | |
| CGE (Non- | Intact IgG | Qualitative | All samples had an increase in |
| Reducing) | Fragment | assessment of | fragment. Adalimumab-Pfizer had |
| | | electropherogram | lower fragments. |
| | | profiles, quantitative | |
| | | assessment of | |
| | | relative change in | |
| | | fragments | |
| CGE | H and L | Qualitative | Reference product had higher |
| (Reducing) | integrity, | assessment of | increase in fragments. |
| | Fragments | electropherogram | |
| | | profiles, quantitative | |
| | | assessment of | |
| | | relative change in | |
| | | fragments | |
| Cell-Based | Relative | Quantitative | Tested |
| Bioassay | Potency | assessment of | |
| | | potency. | |
| HIAC^{b} | Sub-visible | Semi-quantitative | All samples showed an increase in |
| | particulates | assessment of sub- | sub-visible particulate matter. |
| | | visible particle levels | Reference drug product had higher |
| | | | particles. |
| LC/MS- | Primary | Identification of | All samples showed peptides H15, |
| Peptide | Structure | potential | and H30 had an increase in |
| Mapping | at Peptide | modifications at the | oxidation. |
| | Level | peptide level. | |

a. Results are summarized in Tables 6-8.

TABLE 6
Summary Table for Comparative Forced Degradation Study for Adalimumab-Pfizer Drug Products

| | | adalimumab-PF | | | adalimumab-PF | | | adalimumab-PF | | |
|---|---|---|---|---|---|---|---|---|---|---|
| Source | | Lot 021B14 | | | Lot 00706646-0045-B | | | Lot 00706646-0045-C | | |
| Lot Number | | | Post- | | | Post- | | | Post- | |
| Analytical | Evaluation | T = 0 | Photoexposure | | T = 0 | Photoexposure | | T = 0 | Photoexposure | |
| Procedure | Parameter | Days | T = 7 Days | | Days | T = 7 Days | | Days | T = 7 Days | |
| Appearance | Visible | EFVP | EFVP | | EFVP | EFVP | | EFVP | EFVP | |
| | Particles | | | | | | | | | |
| | Clarity | I | I | | I | I | | I | I | |
| | Coloration | B9 | B6 | | B9 | B6 | | B9 | B6 | |
| | | | Post- | | | Post- | | | Post- | |
| | | | Photo- | | | Photo- | | | Photo- | |
| Analytical | Evaluation | T = 0 | exposure | Change | T = 0 | exposure | Change | T = 0 | exposure | Change |
| Procedure | Parameter | Days | T = 7 Days | from T0 | Days | T = 7 Days | from T0 | Days | T = 7 Days | from T0 |
| UV | Protein | 49.0 | 49.0 | 0.0 | 47.9 | 48.1 | 0.2 | 50.0 | 50.3 | 0.3 |
| spectro- | Concentra- | | | | | | | | | |
| scopy | tion | | | | | | | | | |
| | (mg/mL) | | | | | | | | | |
| pH | pH | 5.54 | 5.50 | −0.04 | 5.54 | 5.51 | −0.03 | 5.55 | 5.50 | −0.05 |
| Methionine | Methionine | 2.2 | 19.2 | 17.0 | 4.1 | 16.8 | 12.7 | 1.8 | 15.4 | 13.6 |
| oxidation | oxidation | | | | | | | | | |
| | (%) | | | | | | | | | |
| iCE | Acidic | 17.9 | 43.0 | 25.1 | 21.1 | 47.0 | 25.9 | 20.0 | 43.7 | 23.7 |
| | Peaks (%) | | | | | | | | | |
| | Main | 60.8 | 39.6 | −21.2 | 65.0 | 41.6 | −23.4 | 62.3 | 41.8 | −20.5 |
| | Peak (%) | | | | | | | | | |
| | Basic | 21.3 | 17.4 | −3.9 | 13.9 | 11.3 | −2.6 | 17.2 | 14.6 | −2.6 |
| | Peaks (%) | | | | | | | | | |
| Size | Monomer (%) | 99.5 | 95.0 | −4.5 | 99.4 | 95.3 | −4.1 | 99.6 | 94.9 | −4.7 |
| Exclusion | HMMS (%) | 0.4 | 3.6 | 3.2 | 0.6 | 3.5 | 2.9 | 0.4 | 3.8 | 3.4 |
| HPLC | LMMS (%) | 0.0 | 1.4 | 1.4 | 0.0 | 11.0 | 11.0 | 0.0 | 1.3 | 1.3 |
| CGE | IgG (%) | 96.8 | 91.7 | −5.1 | 97.4 | 90.5 | −6.9 | 97.7 | 91.4 | −6.3 |
| (Non- | Fragment (%) | 3.2 | 6.8 | 3.6 | 2.6 | 7.9 | 5.3 | 2.3 | 7.0 | 4.7 |
| reducing) | Other (%) | 0.0 | 1.5 | 1.5 | 0.0 | 1.6 | 1.6 | 0.0 | 1.5 | 1.5 |
| CGE | Heavy | 99.1 | 97.7 | −1.4 | 99.2 | 97.5 | −1.7 | 99.2 | 97.6 | −1.6 |
| (reducing) | Chain + Light | | | | | | | | | |
| | Chain (%) | | | | | | | | | |
| | Fragment (%) | 0.3 | 0.0 | −0.3 | 0.3 | 0.0 | −0.3 | 0.3 | 0.0 | −0.3 |
| | Other (%) | 0.6 | 2.3 | 1.7 | 0.5 | 2.5 | 2.0 | 0.5 | 2.4 | 1.9 |
| Bioassay | Relative | T | T | T | T | T | T | T | T | T |
| | Potency (%) | | | | | | | | | |
| Sub- | ≧10 μm | 215.0 | 170.0 | −45.0 | 296.3 | 351.3 | 55.0 | 285.5 | 175.7 | −109.8 |
| visible | per mL | | | | | | | | | |
| Particles | ≧25 μm | 1.67 | 1.67 | 0.00 | 5.67 | 4.00 | −1.67 | 2.00 | 4.67 | 2.67 |
| | per ML | | | | | | | | | |

TABLE 7
Summary Table for Comparative Forced Degradation Study for Adalimumab-US Drug Products

| | | Reference Product-US | | | Reference Product-US | | | Reference Product-US | | |
|---|---|---|---|---|---|---|---|---|---|---|
| Source | | Lot 1010535 | | | Lot 1010534 | | | Lot 1010847 | | |
| Lot Number | | | Post- | | | Post- | | | Post- | |
| Analytical | Evaluation | T = 0 | Photoexposure | | T = 0 | Photoexposure | | T = 0 | Photoexposure | |
| Procedure | Parameter | Days | T = 7 Days | | Days | T = 7 Days | | Days | T = 7 Days | |
| Appearance | Visible | EFVP | EFVP | | EFVP | EFVP | | EFVP | EFVP | |
| | Particles | | | | | | | | | |
| | Clarity | IV | IV | | IV | IV | | IV | IV | |
| | Coloration | B9 | B6 | | B9 | B6 | | B9 | B6 | |
| | | | Post- | | | Post- | | | Post- | |
| | | | Photo- | | | Photo- | | | Photo- | |
| Analytical | Evaluation | T = 0 | exposure | Change | T = 0 | exposure | Change | T = 0 | exposure | Change |
| Procedure | Parameter | Days | T = 7 Days | from T0 | Days | T = 7 Days | from T0 | Days | T = 7 Days | from T0 |
| UV | Protein | 47.5 | 47.1 | −0.4 | 47.0 | 47.1 | 0.1 | 48.4 | 47.8 | −0.6 |
| spectro- | Concentra- | | | | | | | | | |
| scopy | tion | | | | | | | | | |
| | (mg/mL) | | | | | | | | | |
| pH | pH | 5.28 | 5.31 | 0.03 | 5.26 | 5.31 | 0.05 | 5.27 | 5.31 | 0.04 |
| Methionine | Methionine | 2.0 | 67.2 | 65.2 | 1.9 | 68.9 | 67.0 | 1.8 | 62.4 | 60.6 |
| oxidation | oxidation | | | | | | | | | |
| | (%) | | | | | | | | | |
| iCE | Acidic | 16.5 | 31.9 | 15.4 | 17.5 | 30.8 | 13.3 | 18.1 | 31.6 | 13.5 |
| | Peaks (%) | | | | | | | | | |
| | Main | 62.3 | 50.8 | −11.5 | 61.2 | 52.2 | −9.0 | 60.8 | 50.8 | −10.0 |
| | Peak (%) | | | | | | | | | |
| | Basic | 21.2 | 17.4 | −3.8 | 21.3 | 17.1 | −4.2 | 21.1 | 17.7 | −3.4 |
| | Peaks (%) | | | | | | | | | |
| Size | Monomer (%) | 99.6 | 86.9 | −12.7 | 99.6 | 87.6 | −12.0 | 99.6 | 87.9 | −11.7 |
| Exclusion | HMMS (%) | 0.3 | 10.9 | 10.6 | 0.3 | 10.9 | 10.6 | 0.3 | 10.1 | 9.8 |
| HPLC | LMMS (%) | 0.1 | 2.2 | 2.1 | 0.1 | 2.5 | 2.4 | 0.1 | 2.1 | 2.0 |
| CGE | IgG (%) | 97.4 | 85.2 | −12.2 | 97.2 | 86.5 | −10.7 | 97.7 | 86.5 | −11.2 |
| (Non- | Fragment (%) | 2.6 | 10.3 | 7.7 | 2.8 | 8.9 | 6.1 | 2.3 | 9.3 | 7.0 |
| reducing) | Other (%) | 0.0 | 4.5 | 4.5 | 0.0 | 4.6 | 4.6 | 0.0 | 4.2 | 4.2 |
| CGE | Heavy | 98.2 | 95.8 | −2.4 | 98.1 | 96.0 | −2.1 | 98.0 | 96.0 | −2.0 |
| (reducing) | Chain + Light | | | | | | | | | |
| | Chain (%) | | | | | | | | | |
| | Fragment (%) | 1.1 | 1.4 | 0.3 | 1.1 | 1.4 | 0.3 | 1.1 | 1.4 | 0.3 |
| | Other (%) | 0.7 | 2.8 | 2.1 | 0.8 | 2.6 | 1.8 | 0.8 | 2.6 | 1.8 |
| Bioassay | Relative | T | T | T | T | T | T | T | T | T |
| | Potency (%) | | | | | | | | | |
| Sub- | ≧10 μm | 2428.0 | 3057.3* | 629.33 | 4517.3 | 4555.0* | 37.67 | 2803.0 | 3008.7* | 205.67 |
| visible | per mL | | | | | | | | | |
| Particles | ≧25 μm | 26.3 | 33.7 | 7.34 | 53.7 | 43.3 | −10.34 | 61.3 | 30.0 | −31.33 |
| | per ML | | | | | | | | | |

TABLE 8
Summary Table for Comparative Forced Degradation Study for Adalimumab-EU Drug Products

| | | Reference Product-EU | | | Reference Product-EU | | | Reference Product-EU | | |
|---|---|---|---|---|---|---|---|---|---|---|
| Source | | Lot 21362XH07 | | | Lot 25365XH04 | | | Lot 33425XD08 | | |
| Lot Number | | | Post- | | | Post- | | | Post- | |
| Analytical | Evaluation | T = 0 | Photoexposure | | T = 0 | Photoexposure | | T = 0 | Photoexposure | |
| Procedure | Parameter | Days | T = 7 Days | | Days | T = 7 Days | | Days | T = 7 Days | |
| Appearance | Visible | EFVP | EFVP | | EFVP | EFVP | | EFVP | EFVP | |
| | Particles | | | | | | | | | |
| | Clarity | IV | IV | | IV | IV | | IV | IV | |
| | Coloration | B9 | B6 | | B9 | B6 | | B9 | B6 | |
| | | | Post- | | | Post- | | | Post- | |
| | | | Photo- | | | Photo- | | | Photo- | |
| Analytical | Evaluation | T = 0 | exposure | Change | T = 0 | exposure | Change | T = 0 | exposure | Change |
| Procedure | Parameter | Days | T = 7 Days | from T0 | Days | T = 7 Days | from T0 | Days | T = 7 Days | from T0 |
| UV | Protein | 48.8 | 48.6 | −0.2 | 48.2 | 48.4 | 0.2 | 48.7 | 48.5 | −0.2 |
| spectro- | Concentra- | | | | | | | | | |
| scopy | tion | | | | | | | | | |
| | (mg/mL) | | | | | | | | | |
| pH | pH | 5.32 | 5.38 | 0.1 | 5.33 | 5.35 | 0.02 | 5.25 | 5.30 | 0.05 |
| Methionine | Methionine | 2.1 | 55.5 | 53.4 | 1.9 | 52.2 | 50.3 | 1.8 | 57.0 | 55.2 |
| oxidation | oxidation | | | | | | | | | |
| | (%) | | | | | | | | | |
| iCE | Acidic | 18.6 | 32.8 | 14.2 | 19.1 | 32.0 | 12.9 | 19.3 | 31.6 | 12.3 |
| | Peaks (%) | | | | | | | | | |
| | Main | 58.0 | 48.8 | −9.2 | 60.1 | 49.4 | −10.7 | 59.5 | 49.4 | −10.1 |
| | Peak (%) | | | | | | | | | |
| | Basic | 23.4 | 18.4 | −5.0 | 20.8 | 18.7 | −2.1 | 21.3 | 19.0 | −2.3 |
| | Peaks (%) | | | | | | | | | |
| Size | Monomer (%) | 99.5 | 84.1 | −15.4 | 99.5 | 86.6 | −12.9 | 99.6 | 89.4 | −10.2 |
| Exclusion | HMMS (%) | 0.4 | 13.8 | 13.4 | 0.4 | 11.2 | 10.8 | 0.3 | 8.5 | 8.2 |
| HPLC | LMMS (%) | 0.1 | 2.1 | 2.0 | 0.1 | 2.2 | 2.1 | 0.1 | 2.1 | 2.0 |
| CGE | IgG (%) | 87.5 | 83.9 | −3.6 | 97.4 | 86.0 | −11.4 | 97.4 | 86.3 | −11.1 |
| (Non- | Fragment (%) | 2.5 | 10.4 | 7.9 | 2.6 | 9.2 | 6.6 | 2.6 | 10.0 | 7.4 |
| reducing) | Other (%) | 0.0 | 5.6 | 5.6 | 0.0 | 4.8 | 4.8 | 0.0 | 3.7 | 3.7 |
| CGE | Heavy | 98.5 | 95.7 | −2.8 | 98.3 | 96.2 | −2.1 | 98.3 | 96.0 | −2.3 |
| (reducing) | Chain + Light | | | | | | | | | |
| | Chain (%) | | | | | | | | | |
| | Fragment (%) | 0.9 | 1.3 | 0.4 | 0.9 | 1.1 | 0.2 | 0.9 | 1.2 | 0.3 |
| | Other (%) | 0.6 | 3.0 | 2.4 | 0.8 | 2.7 | 1.9 | 0.7 | 2.6 | 1.9 |
| Bioassay | Relative | T | T | T | T | T | T | T | T | T |
| | Potency (%) | | | | | | | | | |
| Sub- | ≧10 μm | 1018.0 | 1340.0* | 322.0 | 2161.0 | 3432.3* | 1271.3 | 1169.0 | 459.3 | −709.7 |
| visible | per mL | | | | | | | | | |
| Particles | ≧25 μm | 25.0 | 3.7 | −21.3 | 84.7 | 13.7 | −71.0 | 6.0 | 4.3 | −1.7 |
| | per ML | | | | | | | | | |

Adalimumab-PF drug product had lower LMMS and HMMS (demonstrated by SEC) and lower fragments (demonstrated by reducing CGE) compared to the adalimumab reference drug products. Higher methionine oxidation was observed for the adalimumab reference drug product compared to adalimumab-PF drug product. Overall, sub-visible particle counts increased after 7 days of photoexposure with the reference drug product lots maximizing the sensors. iCE showed higher % acidic species for adalimumab-PF compared to the reference drug products. However, no new species were observed for the adalimumab-PF product compared to the reference products. The non-reducing CGE showed less fragments for adalimumab-PF drug product compared to the reference drug products. All reference product lots performed similar to each other.

The LC/MS analysis of the Lys-C peptide maps showed that peptides H15 and H30 were the only peptides that were modified in the T=7 day samples. For these peptides, an increase in oxidation was observed for all materials, likely corresponding to oxidation at Met²⁵⁶ of the H15 peptide and Met⁴³² of the H30 peptide. In all cases, the chromatographic profiles for the T=7 samples were compared with T=0 day in terms of peak elution positions, peak shapes and found similar with no other changes. Adalimumab-PF had lower oxidation compared to reference product.

In summary, no new degradation species were observed in the adalimumab-PF drug products when compared to the adalimumab reference products. The reference product lots trended similarly across lots. The non-reducing CGE showed consistent trends in the fragment levels across multiple lots eluting to syringe-to-syringe variability as the cause of the results observed in the Part 1 study. Adalimumab-PF drug product lots performed similarly/better compared to the reference drug product with the exception of acidic species (demonstrated by iCE).

It is claimed:

1. An aqueous formulation comprising:
about 35 mg/ml to about 200 mg/ml of an anti-Tumor Necrosis Factor alpha (TNFα) antibody, or an antigen-binding fragment thereof;
a buffer;
a polyol;
methionine;
a surfactant;
a chelating agent; and
wherein the formulation has a pH at about 5.0 to about 6.0.

2. The aqueous formulation of claim 1, wherein the buffer is a histidine buffer.

3. The aqueous formulation of claim 1, wherein the concentration of the buffer is about 1 mM to about 100 mM.

4. The aqueous formulation of claim 1, wherein the polyol is sucrose.

5. The aqueous formulation of claim 1, wherein the concentration of the polyol is about 1 mg/mL to about 300 mg/mL.

6. The aqueous formulation of claim 1, wherein the surfactant is a polysorbate.

7. The aqueous formulation of claim 6, wherein the polysorbate is polysorbate 80 (PS80).

8. The aqueous formulation of claim 1, wherein the concentration of the surfactant is about 0.01 mg/ml to about 10 mg/ml.

9. The aqueous formulation of claim 1, wherein the chelating agent is disodium EDTA (ethylenediaminetetracetic acid) dihydrate.

10. The aqueous formulation of claim 1, wherein the concentration of the chelating agent is about 0.01 mg/ml to about 1.0 mg/ml.

11. The aqueous formulation of claim 1, wherein the antibody, or the antigen-binding fragment thereof, comprises a heavy chain variable region (VH) complementarity determining region one (CDR1) having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 3 or 12, or a variant of SEQ ID NO: 3 having a single alanine substitution at position 2, 3, 4, 5, 6, 8, 9, 10, or 11, or by one to five conservative amino acid substitutions at positions 2, 3, 4, 5, 6, 8, 9, 10, 11, and/or 12, and a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 6 or 13, or a variant of SEQ ID NO: 6 having a single alanine substitution at position 1, 4, 5, 7, or 8, or by one to five conservative amino acid substitutions at positions 1, 3, 4, 6, 7, 8, and/or 9.

12. The aqueous formulation of claim 1, wherein the antibody, or the antigen-binding fragment thereof, comprises a heavy chain variable region (VH) and a light chain variable region (VL), wherein the VH region comprises the amino acid sequence of SEQ ID NO: 7, and the VL region comprises the amino acid sequence of SEQ ID NO: 8.

13. The aqueous formulation of claim 1, wherein the antibody, or the antigen-binding fragment thereof, comprises a heavy chain variable region comprising the CDR1, CDR2, and CDR3 of adalimumab, and a light chain variable region CDR1, CDR2, and CDR3 of adalimumab.

14. (canceled)

15. The aqueous formulation of claim 1, wherein the formulation has a shelf life of at least about 24 months.

16. An aqueous formulation comprising:
about 35 mg/ml to about 200 mg/ml of an anti-Tumor Necrosis Factor alpha (TNFα) antibody, or an antigen-binding fragment thereof;
about 1 mM to about 100 mM of a buffer;
about 1 mg/mL to about 300 mg/mL of a polyol;
about 0.01 mg/mL to about 10 mg/mL of methionine;
about 0.01 mg/ml to about 10 mg/ml of a surfactant;
about 0.001 mg/ml to about 1.0 mg/ml of a chelating agent; and
wherein the formulation has a pH at about 5.0 to about 6.0.

17. An aqueous formulation comprising:
about 35 mg/ml to about 200 mg/ml of an anti-Tumor Necrosis Factor alpha (TNFα) antibody, or an antigen-binding fragment thereof;
about 1 mM to about 100 mM of a buffer;
about 1 mg/mL to about 300 mg/mL of a polyol;
about 0.01 mg/mL to about 10 mg/mL of methionine;
about 0.01 mg/ml to about 10 mg/ml of a surfactant;
about 0.01 mg/ml to about 1.0 mg/ml of a chelating agent;
wherein the formulation has a pH at about 5.0 to about 6.0; and wherein the antibody, or the antigen-binding fragment thereof, comprises a heavy chain variable region (VH) complementarity determining region one (CDR1) having the amino acid sequence shown in SEQ ID NO: 1 or 10, a VH CDR2 having the amino acid sequence shown in SEQ ID NO: 2 or 11, a VH CDR3 having the amino acid sequence shown in SEQ ID NO: 3 or 12, or a variant of SEQ ID NO: 3 having a single alanine substitution at position 2, 3, 4, 5, 6, 8, 9, 10, or 11, or by one to five conservative amino acid substitutions at positions 2, 3, 4, 5, 6, 8, 9, 10, 11, and/or 12, and a light chain variable region (VL) CDR1 having the amino acid sequence shown in SEQ ID NO: 4, a VL CDR2 having the amino acid sequence shown in SEQ ID NO: 5, and a VL CDR3 having the amino acid sequence shown in SEQ ID NO: 6 or 13, or a variant of SEQ ID NO: 6 having a single alanine substitution at position 1, 4, 5, 7, or 8, or by one to five conservative amino acid substitutions at positions 1, 3, 4, 6, 7, 8, and/or 9.

18. (canceled) The aqueous formulation of claim 17, wherein the antibody is adalimumab.

19. The aqueous formulation of claim 1, wherein the concentration of the antibody, or the antigen-binding fragment thereof, is 35 mg/mL, 40 mg/mL, 45 mg/ml, 50 mg/mL, 55 mg/mL, or 60 mg/mL.

20. An aqueous formulation comprising:
50 mg/ml of an anti-Tumor Necrosis Factor alpha (TNFα) protein, or an antigen-binding fragment thereof;
about 20 mM histidine buffer;
about 85 mg/mL sucrose;
about 0.2 mg/mL methionine;
about 0.2 mg/ml polysorbate 80;
about 0.05 mg/ml disodium EDTA dihydrate;
wherein the antibody, or the antigen-binding fragment thereof, comprises a heavy chain variable region comprising the amino acid sequence of SEQ ID NO: 7, and a light chain variable region comprising the amino acid sequence of SEQ ID NO: 8; and
wherein the formulation has pH at 5.5.

21. (canceled)

22. The aqueous formulation of claim 16, wherein the formulation has a shelf life of at least about 24 months.

23. A method for treating or inhibiting a TNFα related disorder in a subject in need thereof, comprising administering to the subject a therapeutically effective amount of the formulation of claim 1.

24. The method of claim 23, wherein the TNFα related disorder is selected from the group consisting of rheumatoid arthritis, juvenile idiopathic arthritis, axial spondyloarthritis, psoriatic arthritis, ankylosing spondylitis, axial spondyloarthritis without radiographic evidence of ankylosing spondylitis, adult Crohn's disease, pediatric Crohn's disease, ulcerative colitis, psoriasis arthropathica, intestinal behcet's disease, plaque psoriasis, and hidradenitis suppurativa.

25. The method of claim 23, wherein the formulation is administered to the subject subcutaneously or intravenously.

26. The method of claim 23, wherein the subject is human

27-28. (canceled)


SEQUENCE LISTING (13 sequences)

SEQ ID NO: 1 - protein, 5 aa, Artificial Sequence
  Synthetic Construct
DYAMH

SEQ ID NO: 2 - protein, 17 aa, Artificial Sequence
  Synthetic Construct
AITWNSGHIDYADSVEG

SEQ ID NO: 3 - protein, 12 aa, Artificial Sequence
  Synthetic Construct
  misc_feature (12)..(12): wherein Xaa can be Tyr or Asn
VSYLSTASSLDX

SEQ ID NO: 4 - protein, 11 aa, Artificial Sequence
  Synthetic Construct
RASQGIRNYLA

SEQ ID NO: 5 - protein, 7 aa, Artificial Sequence
  Synthetic Construct
AASTLQS

SEQ ID NO: 6 - protein, 9 aa, Artificial Sequence
  Synthetic Construct
  misc_feature (9)..(9): wherein Xaa can be Thr or Ala
QRYNRAPYX

SEQ ID NO: 7 - protein, 121 aa, Artificial Sequence [sequence omitted]

SEQ ID NO: 8 - protein, 107 aa, Artificial Sequence [sequence omitted]

SEQ ID NO: 9 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 10 - protein, 7 aa, Artificial Sequence
  Synthetic Construct
GFTFDDY

SEQ ID NO: 11 - protein, 6 aa, Artificial Sequence
  Synthetic Construct
TWNSGH

SEQ ID NO: 12 - protein, 12 aa, Artificial Sequence
  Synthetic Construct
VSYLSTASSLDY

SEQ ID NO: 13 - protein, 9 aa, Artificial Sequence
  Synthetic Construct
QRYNRAPYT